(19) United States
(12) Patent Application Publication
(10) Pub. No.: US 2025/0034146 A1
(43) Pub. Date: Jan. 30, 2025

(54) ELECTRO-PHOTOCHEMICAL SYNTHESIS OF 1,2,4-TRIAZOLO-[4,3-A]PYRAZINE

(71) Applicant: Purdue Research Foundation, West Lafayette, IN (US)

(72) Inventors: Davin Glenn Piercey, Lafayette, IN (US); Joseph Robert Yount, Lafayette, IN (US)

(73) Assignee: Purdue Research Foundation, West Lafayette, IN (US)

(21) Appl. No.: 18/661,871

(22) Filed: May 13, 2024

Related U.S. Application Data
(60) Provisional application No. 63/526,567, filed on Jul. 13, 2023.

(51) Int. Cl. C07D 487/04

(52) U.S. Cl. CPC C07D 487/04

(57) ABSTRACT

A method of preparing a 1,2,4-triazolo-[4,3-a]pyrazine backbone using an electro-photochemical process comprising coupling a compound comprising a 5-substituted tetrazole moiety to a compound comprising an optionally substituted pyrazine moiety electrochemically and subjecting the resulting compound to photochemical excitation with ultraviolet light.

### CROSS-REFERENCE TO RELATED APPLICATION

This application claims priority to U.S. provisional patent application No. 63/526,567, which was filed Jul. 13, 2023, and which is hereby incorporated by reference in its entirety.

### STATEMENT OF GOVERNMENT SUPPORT

This invention was made with government support under W911NF2020189 awarded by the Army Research Office. The government has certain rights in the invention.

### TECHNICAL FIELD

The present disclosure relates to a method for preparing 1,2,4-triazolo-[4,3-a]pyrazines utilizing an electro-photochemical process and the use of the compounds as scaffolding for the synthesis of energetic materials.

### BACKGROUND

Nitrogen-rich annulated heterocycles are of vital importance within nature and are also widely used in many manufactured goods. The high natural prevalence of bioactive N-heterocycles within nature makes them a convenient building block for drug discovery. For example, purine is the naturally most abundant N-heterocycle and is vital to all life on Earth. The purine family plays an important role in inflammation regulation throughout the human body by activating adenosine receptors (ARs)(A. Marti Navia et al., Cells, 2020, 9, 1739). Purine is also the molecular backbone of the world's most commonly consumed psychoactive substance, caffeine, and its cocoa congener, theobromine.

Nitrogen-rich annulated heterocycles have also been used as energetic materials in potential explosives, propellants, or pyrotechnics. Heterocycles with a high nitrogen content are attractive in these fields due to their higher heats of formation, their increased densities, and their improved oxygen balances in comparison to their high-carbon energetic counterparts (Pagoria et al., Thermochim Acta, 2002, 384, 187-204). There is also an environmental motivation for the use of nitrogen-rich materials. These materials on combustion afford mostly nontoxic nitrogen gas, in contrast to the high levels of soot, carbon monoxide, and carbon dioxide produced by carbon-rich materials (Qu et al., Journal of Material Chemistry A, 2018, 6,1915-1940).

One annulated N-heterocycle, which has gained attention as a therapeutic drug molecule and as a potential energetic scaffold, is the 1,2,4-triazolo-[4,3-a]pyrazine. It has a variety of biological applications, such as use in the backbone of blood pressure medication and anti-cancer, anti-malarial, anti-viral, and anti-microbial applications. For example, a promising use for 1,2,4-triazolo-[4,3-a]pyrazine is the design of A₂ₐAR, one of four adenosine receptor antagonists (Falsini et al., Journal of Medicinal Chemistry, 2017, 60, 5772-5790). A₂ₐAR antagonists may have therapeutic significance for neurodegenerative diseases such as Huntington's disease and Parkinson's disease. The functional groups present on the 1,2,4-triazolo-[4,3-a]pyrazine are important to determine the molecule's therapeutic efficacy. Tailored functionalization can impart nanomolar affinities with complete selectivity to specific adenosine receptor subtypes.

1,2,4-triazolo-[4,3-a]pyrazines are generally prepared via a two-step procedure in which (i) a suitable chloropyrazine is reacted with hydrazine at elevated temperatures to yield a hydrazinopyrazine and (ii) cyclization of a hydrazinopyrazine using orthoesters to form the 1,2,4-triazolo-[4,3-a]pyrazine. This procedure requires the use of hydrazine and elevated reaction temperatures, as well as a chlorinated starting material. A known alternative method involves a five-step procedure starting with the reaction of ethyl 2-chloroacetoacetate and an aryldiazonium chloride (M. Falsini et al., Journal of Medicinal Chemistry, 2017, 60, 5772-5790). The method requires the formation of energetically sensitive diazonium salts and is a more costly and environmentally burdensome multi-step procedure.

Hence, there is a need for a method for the preparation of 1,2,4-triazolo-[4,3-a]pyrazines, which is cost-effective, short, and eco-friendly. It is an object of the present disclosure to provide such a method. This and other objects and advantages, as well as inventive features, will be apparent from the detailed description.

### SUMMARY

Provided is a method for preparing a 1,2,4-triazolo-[4,3-a]pyrazine of formula (IV):

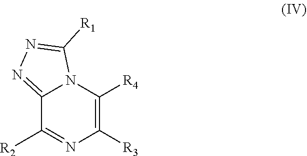

wherein,
each R₁, R₂, R₃, and R₄ is independently hydrogen, halogen, nitrile, azide, nitro, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X is O or S;
each R₅ and R₆ is independently hydrogen or a substituted or an unsubstituted group selected from alkyl, trifluoroalkyl, cycloalkyl, hydroxyl, alkoxy, thiol, thioalkyl, amine, aminoalkyl, carboxy, aminocarbonyl, N-alkyl aminocarbonyl, N,N-dialkyl aminocarbonyl, heteroalkyl, heterocycloalkyl, aryl, heteroaryl, heteroarylalkyl, heteroalkylaryl, arylalkenyl, and arylalkyl;
which method comprises:
(i) mixing a compound of formula (I):

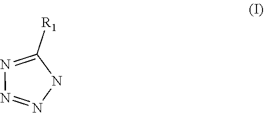

wherein,
R₁ is hydrogen, halogen, nitrile, azide, nitro, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X is O or S;
each R₅ and R₆ is independently hydrogen or a substituted or an unsubstituted group selected from alkyl, trifluoroalkyl, cycloalkyl, hydroxyl, alkoxy, thiol, thioalkyl, amine, aminoalkyl, carboxy, aminocarbonyl, N-alkyl aminocarbonyl, N,N-dialkyl aminocarbonyl, heteroalkyl, heterocycloalkyl, aryl, heteroaryl, heteroarylalkyl, heteroalkylaryl, arylalkenyl, and arylalkyl;
with a compound of formula (II):

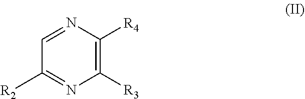

wherein,
each R₂, R₃, and R₄ is independently hydrogen, halogen, nitrile, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, aryl, heteroaryl, heteroarylalkyl, heteroalkylaryl, arylalkenyl, and arylalkyl, wherein X, R₅, and R₆ are as defined above;
in the presence of an organic solvent and an electrolyte to obtain a reaction mixture;
(ii) subjecting the reaction mixture of step (i) to an electrolysis to obtain a compound of formula (III):

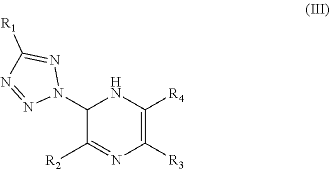

wherein,
each R₁, R₂, R₃, and R₄ is independently hydrogen, halogen, nitrile, azide, nitro, or a substituted or an unsubstituted group selected from alkyl, trifluoroalkyl, cycloalkyl, X—R₅,
NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X, R₅, and R₆ are as defined above; and
(iii) subjecting the compound of formula (III) to a photochemical reaction using an ultraviolet (UV) light,
whereupon 1,2,4-triazolo-[4,3-a]pyrazine of formula (IV) is obtained.

In some embodiments, R₁ is hydrogen or halogen and R₄ is hydrogen.

In some embodiments, R₁ is hydrogen, halogen, azide (N₃), nitro or a substituted or an unsubstituted group selected from alkyl, cycloalkyl, OR₅, SR₅, NR₅R₆, aryl, and heterocyclyl, wherein each R₅ and R₆ is independently selected from hydrogen, alkyl, trifluoroalkyl, cycloalkyl, aryl, heteroaryl, heterocyclyl, heteroarylalkyl, heteroalkylaryl, arylalkenyl, and arylalkyl. In some embodiments, R₁ is selected from hydrogen, halogen, N₃, OMe, S-Et, methyl, isovaleryl, cyclopropyl, NO₂, CH₂—NO₂, NH₂, Phenyl, tetrazole, furan, 2-nitro furan, benzyl, difluoro benzyl and difluoroazetidine.

In some embodiments, the compound of formula (II) is a compound of formula (HA):

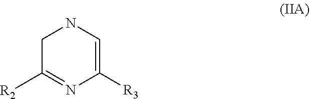

wherein,
each R₂ and R₃ is independently hydrogen, halogen, nitrile, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, aryl, heteroaryl, heteroarylalkyl, heteroalkylaryl, arylalkenyl, and arylalkyl, wherein X, R₅, and R₆ are as defined above.

In some embodiments, each R₂ and R₃ is independently hydrogen, halogen or a substituted or an unsubstituted group selected from alkyl and OR₅, wherein R₅ is alkyl, trifluoroalkyl, cycloalkyl, aryl, heteroaryl, heterocyclyl, heteroarylalkyl, heteroalkylaryl, arylalkenyl, or arylalkyl.

The electrolyte used in step (i) can be selected from tetraalkylammonium salts, trialkyl ammonium salts, tetraalkylammonium tetrazolate, organic or inorganic perchlorates, organic or inorganic tetrafluoroborates, organic or inorganic nitrates, and organic or inorganic hexafluorophosphates. In some embodiments, the electrolyte is selected from tetraethylammonium tetrafluoroborate, sodium tetrafluoroborate, sodium perchlorate, tetraethylammonium perchlorate, tetrapropylammonium perchlorate, tetraethylammonium tetrazolate, tetraethylammonium bromo tetrazolate, tetrabutylammonium hexafluorophosphate and lithium nitrate.

The photochemical reaction can be carried out using a UV light having a wavelength of about 155 nm to about 465 nm. Desirably, the wavelength is of about 250 nm to about 370 nm. The photochemical reaction can be carried out using an organic solvent.

Any suitable organic solvent can be used in the eletrolysis process and photochemical process. In some embodiments, the organic solvent is a polar aprotic organic solvent.

Also provided is a method for synthesizing an energetic material 5-nitro-[1,2,4]triazolo[4,3-a]pyrazine-6,8-diamine of formula (27), which method comprises:

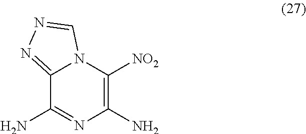 [SMILES: Nc1nc(N)c2nncn2c1[N+](=O)[O-]]

(i) preparing 6,8-dimethoxy-1,2,4-triazolo-[4,3-a]pyrazine a compound of formula (15a), wherein the compound of formula (15a) is prepared according to the above-described method; and

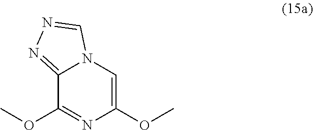

(ii) introducing a nitro and an amino group to the compound of formula (15a), whereupon the energetic material 5-nitro-[1,2,4]triazolo[4,3-a]pyrazine-6,8-diamine of formula (27) is synthesized.

### BRIEF DESCRIPTION OF THE DRAWINGS

The present disclosure will be more readily understood from the detailed description of embodiments presented below, considered in conjunction with the attached drawings of which:

FIG. 1A shows the cyclic voltammogram of 1H-tetrazole (3.3 mM) in dry acetonitrile, and the electrolyte used is 100 mM tetraethylammonium perchlorate.

FIG. 1B shows the cyclic voltammogram of 5-bromotetrazole (1.9 mM) in dry acetonitrile, and the electrolyte used is 100 mM tetraethylammonium perchlorate.

FIG. 1C. shows the cyclic voltammogram of 2,6-dimethoxypyrazine (2.2 mM) in dry acetonitrile, and the electrolyte used is 100 mM tetraethylammonium perchlorate.

FIG. 2 shows the thermogravimetric isotherm of 5-nitro-[1,2,4]triazolo[4,3-a]pyrazine-6,8-diamine (27), an energetic material. Heated at 5° C./min in an alumina crucible under argon gas.

FIG. 3 shows the UV absorption spectra of compound (10), compound (13a), and compound (13_{b}).

FIG. 4 shows the UV absorption spectra of compound (10), compound (11a), compound (11b), compound (12a), compound (12b), compound (13a), and compound (13b).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term “energetic material” refers to a class of material with a high amount of stored chemical energy that can be released. For example, explosives, pyrotechnic compositions, propellants, and fuels.

Abbreviations used for the chemical groups are: Me is methyl, Et is ethyl, Ph is pehnyl, benzyl is CH₂-Ph, and acetonitrile is MeCN.

The present disclosure is predicated, at least in part, on the discovery that 1,2,4-triazolo[4,3-b]pyridazine isomer of 1,2,4-triazolo-[4,3-a]pyrazines has been used to produce high-performing, insensitive energetics. 1,2,4-triazolo[4,3-b]pyridazine are reported to be prepared by the cyclization of hydrazine-substituted heterocycles, such as 3-chloro-6-hydrazineyl-5-nitropyridazin-4-amine with cyanogen bromide (S. Chen et al., Chemical Engineering Journal 2021, 421, 129635). The obtained energetic material, 3,8-dinitro-[1,2,4]triazolo[4,3-b]pyridazine-6,7-diamine, exhibits high density (1.93 g/cm⁻³), high thermal stability (234° C.), insensitivity to friction (>360 N), low sensitivity to impact (20 J), high predicted detonation velocity (˜9000 m/s), and high predicted detonation pressure (34 GPa).

In view of the above, provided is a sustainable method to prepare 1,2,4-triazolo-[4,3-a]pyrazines as a scaffold for synthesizing energetic materials using green chemistry. The method involves electrochemistry, which utilizes the polarization of an electrode to drive chemical reactions via the direct transfer of electrons. The direct use of electrons, rather than chemical oxidation/reduction agents, is referred to as a “green” chemical technique because it aids in reducing waste production by eliminating the need for chemical oxidants/reductants (Horn et al., ACS Century Science, 2016, 2, 302-308). This environmentally friendly method comprises an electrochemical coupling of a compound comprising a tetrazole moiety with a compound comprising an optionally substituted pyrazine moiety, followed by ultraviolet (UV) cyclization to obtain a compound comprising 1,2,4-triazolo-[4,3-a]pyrazine moiety.

Thus, provided is a method for preparing a 1,2,4-triazolo-[4,3-a]pyrazine of formula (IV):

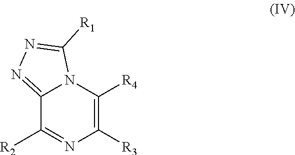

wherein,
each R₁, R₂, R₃, and R₄ is independently hydrogen, halogen, nitrile, azide, nitro, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X is O or S;
each R₅ and R₆ is independently hydrogen or a substituted or an unsubstituted group selected from alkyl, trifluoroalkyl, cycloalkyl, hydroxyl, alkoxy, thiol, thioalkyl, amine, aminoalkyl, carboxy, aminocarbonyl, N-alkyl aminocarbonyl, N,N dialkyl aminocarbonyl, heteroalkyl, heterocycloalkyl, aryl, heteroaryl, heteroarylalkyl, heteroalkylaryl, arylalkenyl, and arylalkyl;
which method comprises:
(i) mixing a compound of formula (I):

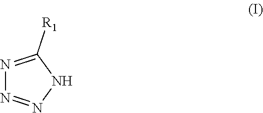

wherein,
R₁ is independently hydrogen, halogen, nitrile, azide, nitro, or a substituted or unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X is O or S;
each R₅ and R₆ is independently hydrogen or a substituted or an unsubstituted group selected from alkyl, trifluoroalkyl, cycloalkyl, hydroxyl, alkoxy, thiol, thioalkyl, amine, aminoalkyl, carboxy, aminocarbonyl, N-alkyl aminocarbonyl, N,N dialkyl aminocarbonyl, heteroalkyl, heterocycloalkyl, aryl, heteroaryl, heteroarylalkyl, heteroalkylaryl, arylalkenyl, and arylalkyl;
with a compound of formula (II):

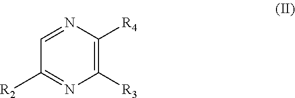

wherein,
each R₂, R₃, and R₄ is independently hydrogen, halogen, nitrile, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, aryl, heteroaryl, heteroarylalkyl, heteroalkylaryl, arylalkenyl, and arylalkyl, wherein X, R₅, and R₆ are as defined above;
in the presence of an organic solvent and an electrolyte to obtain a reaction mixture;
(ii) subjecting the reaction mixture of step (i) to an electrolysis to obtain a compound of formula (III):

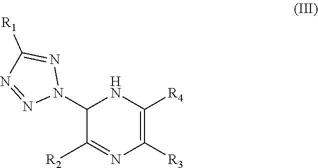

wherein,
each R₁, R₂, R₃, and R₄ is independently hydrogen, halogen, nitrile, azide, nitro, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X, R₅, and R₆ are as defined above; and
(iii) subjecting the compound of formula (III) to a photochemical reaction using an ultraviolet (UV) light,
whereupon 1,2,4-triazolo-[4,3-a]pyrazine of formula (IV) is obtained.

The method for preparing 1,2,4-triazolo-[4,3-a]pyrazine backbone can comprise two steps: an electrolysis process and a photochemical process, respectively. In the first step, a compound comprising 5-substituted tetrazole moiety can be electrochemically coupled to a compound comprising an optionally substituted pyrazine moiety, such as 2,6-disubstituted pyrazine, to yield a compound comprising 2,5-disubstituted tetrazole along with its isomer, a compound comprising 1,5-disubstituted tetrazole as a side product. The electrochemical coupling can produce a mixture of isomers with an isomer ratio dependent upon the substituent at the 5-position of tetrazole or the substituents on the pyrazine. The subsequent second step can comprise a photochemical excitation of the compound comprising 2,5-disubstituted tetrazole using a UV light. The photochemical excitation of the compound can result in the formation of a nitrilimine intermediate with the release of nitrogen gas. The nitrilimine intermediate is a short-lived intermediate that can immediately undergo a cyclization. Rapid cyclization of the nitrilimine intermediate can result in the formation of a compound comprising 1,2,4-triazolo-[4,3-a]pyrazine.

In some embodiments, R₁ is hydrogen or halogen. In some embodiments, R₁ is hydrogen, halogen, azide, nitro, or a substituted or an unsubstituted group selected from alkyl, cycloalkyl, OR₅, SR₅, NR₅R₆, aryl, and heterocyclyl, wherein each R₅ and R₆ is independently selected from hydrogen, alkyl, trifluoroalkyl, cycloalkyl, aryl, heteroaryl, heterocyclyl, heteroarylalkyl, heteroalkylaryl, arylalkenyl, and arylalkyl. In some embodiments, R₁ is selected from hydrogen, halogen, azide (N₃), OMe, S-Et, methyl, isovaleryl, cyclopropyl, NO₂, CH₂—NO₂, NH₂, Phenyl, tetrazole, furan, 2-nitro furan, benzyl, difluoro benzyl, and difluoroazetidine.

In some embodiments, R₄ is hydrogen.

The pyrazine moiety can be optionally substituted. In some embodiments, the pyrazine moiety can be unsubstituted. In some embodiments, the pyrazine moiety can be substituted, e.g., monosubstituted, disubstituted, or trisubstituted. Desirably, the pyrazine ring is disubstituted and represented by formula (HA):

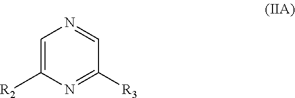

wherein each R₂ and R₃ is independently hydrogen, halogen, nitrile, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, aryl, heteroaryl, heteroarylalkyl, heteroalkylaryl, arylalkenyl, and arylalkyl, wherein X, R₅, and R₆ are as defined above.

In some embodiments, each R₂ and R₃ in the compound of formula (IIA) and (III) is independently hydrogen, a substituted or an unsubstituted alkyl or X—R₅, wherein X is O and R₅ is alkyl. Preferably, each R₂ and R₃ is independently a substituted or an unsubstituted O-alkyl.

In some embodiments, in the compound of formula (IV), R₁ is hydrogen or halogen; Ra is hydrogen; and each R₂ and R₃ is independently hydrogen, a substituted or an unsubstituted alkyl or X—R₅, wherein X is O and R₅ is alkyl. Preferably, each R₂ and R₃ is independently a substituted or an unsubstituted O-alkyl.

A compound comprising 1,5-disubstituted tetrazole moiety of formula (III′) is obtained as a side product in the electrolysis process. The compound of formula (III′) is an isomer of the compound of formula (III):

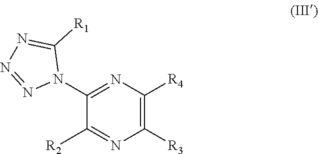

wherein,
each R₁, R₂, R₃ and R₄ are as defined above.

In some embodiments, the photochemical process of the compound of formula (III) can be carried out in the presence of an organic solvent.

Any suitable organic solvent can be used in the photochemical process. In some embodiments, the organic solvent is a polar aprotic organic solvent. Examples of suitable organic solvents used in the photochemical process include, but are not limited to, acetone, ethyl acetate, dimethyl sulfoxide, N,N-dimethyl formamide, dichloromethane, acetonitrile, propionitrile, and tetrahydrofuran. Desirably, the organic solvent is acetonitrile.

The UV light used for the photochemical reaction can be generated from any suitable UV light source, such as a UV lamp. The UV lamp used can be a thin-layer chromatography (TLC) UV lamp. In some embodiments, the wavelength at which the photochemical process was carried out is about 150 nm to about 465 nm, such as about 150 nm to 465 nm, 150 nm to about 465 nm, or 150 nm to 465 nm. The process yield can be controlled by adjusting the wavelength of UV light and either shortening or extending its exposure time, which can either prevent product decomposition or aid in the complete conversion of the starting material, depending on the material of interest.

In some embodiments, the wavelength of UV light is about 254 nm to about 365 nm (such as 254 nm to 365 nm, about 254 nm to 365 nm, or 254 nm to about 365 nm). The time required for irradiation can be about 24 hours to about 48 hours (such as about 24 hours to 48 hours, 24 hours to about 48 hours, 24 hours to 48 hours, 24 hours to 36 hours, or 36 hours to 48 hours), based on the wavelength of the UV lamp used. The shorter wavelength of the UV lamp, such as 254 nm, can decrease the absorption of UV light by the pyrazine ring and decrease the rate of decomposition/photoisomerization.

Any suitable organic solvent can be used in the electrolysis process. In some embodiments, the organic solvent is a polar aprotic organic solvent. Examples of organic solvents used in the electrolysis process include, but are not limited to, acetone, ethyl acetate, dimethyl sulfoxide, N,N-dimethyl formamide, dichloromethane, acetonitrile, propionitrile, and tetrahydrofuran. Desirably, the organic solvent is acetonitrile.

The electrolysis can be carried out using a suitable current value, such as from about 0.01 mA to 100 A. In some embodiments, the electrolysis can be carried out using the current from about 5 mA to about 25 mA, such as from about 5 mA to 25 mA, 5 mA to about 25 mA, or 5 mA to 25 mA. Desirably, the electrolysis can be carried out using a current from about 7.5 mA to about 15 mA, such as from about 7.5 mA to 15 mA, 7.5 mA to about 15 mA, or 7.5 mA to 15 mA. Desirably, the electrolysis can be carried out using a current of about 7.5 mA (such as 7.5 mA).

The electrolysis can be carried out by passing a suitable optimal charge, such as from about 0.01 F/mol to 50 F/mol. In some embodiments, the optimal charge passed during the electrolysis can be from about 2 F/mol to about 3 F/mol, such as from about 2 F/mol to 3 F/mol, 2 F/mol to about 3 F/mol, or 2 F/mol to 3 F/mol. Desirably, the optimal charge passed during the electrolysis can be about 2.1 F/mol (such as 2.1 F/mol).

Any suitable electrolyte can be used. Examples of the electrolytes include, but are not limited to, tetraalkylammonium salts, trialkylammonium salts, tetraalkylammonium tetrazolate organic or inorganic perchlorates, organic or inorganic tetrafluoroborates, organic or inorganic nitrates, and organic or inorganic hexafluorophosphates. In some embodiments, the electrolyte is selected from tetraethylammonium tetrafluoroborate, sodium tetrafluoroborate, sodium perchlorate, tetraethylammonium perchlorate, tetrapropylammonium perchlorate, tetraethylammonium tetrazolate, tetraethylammonium 5-bromo tetrazolate, tetrabutylammonium hexafluorophosphate and lithium nitrate.

In some embodiments, the concentration of electrolyte used can be from about 100 mM to about 300 mM, such as from about 100 mM to 300 mM, 100 mM to about 300 mM, or 100 mM to 300 mM. Desirably, the concentration of electrolyte used is 200 mM.

In the electrolysis process, any suitable electrodes can be used. The electrodes used can be a working electrode, a counter electrode, and a reference electrode. In some embodiments, the reference electrode can be Ag/Ag⁺. In some embodiments, the working electrode and counter electrode can be the same or different. Examples of the working electrodes and the counter electrodes include, but are not limited to, carbon, glassy carbon, platinum, iridium, gold, boron-doped diamond, iron, nickel, stainless steel, lead dioxide, copper, gold, zinc, and titanium. In some embodiments, the electrodes used are platinum (Pt).

Scheme 1 illustrates a method for the preparation of a 3-substituted 1,2,4-triazolo-[4,3-a]pyrazine backbone.

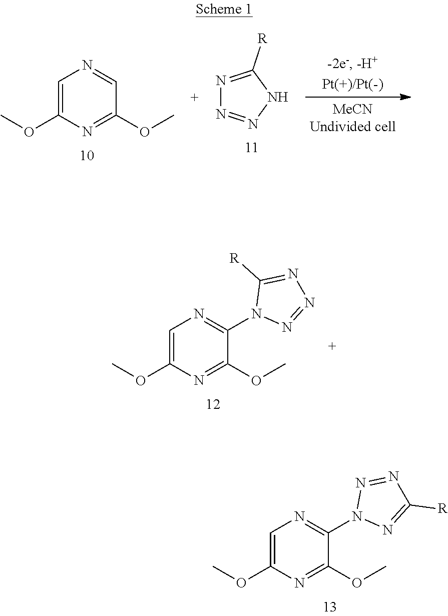

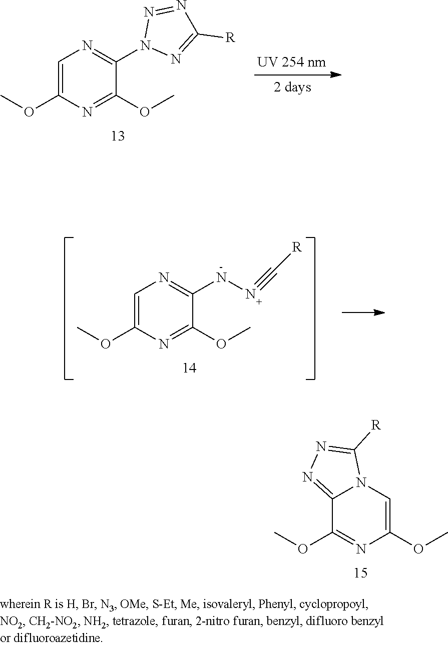

The method comprises: (i) coupling electrochemically a compound of formula (11) to 2,6-dimethoxypyrazine of formula (10) to yield 2,5-disubstituted tetrazole of formula (13) with 1,5-disubstituted tetrazole of formula (12) in which the tetrazole couples at either the N1 or N2 position respectively; (ii) subjecting 2,5-disubstituted tetrazole of formula (13) to a photochemical excitation using a UV lamp with a dual wavelength of 254 nm and/or 365 nm to form a nitrilimine intermediate of formula (14) with the release of nitrogen gas which undergoes cyclization, such as 1,3-dipolar cyclization to obtain 3-substituted 1,2,4-triazolo-[4,3-a]pyrazine of formula (15). The nitrilimine intermediate formed is not a stable product and can undergo cyclization immediately.

In some embodiments, 5H-tetrazole (R is H) couples to the 2,6-dimethoxypyrazine to form a mixture of two isomers. 5H-tetrazole can couple at either its N1 or N2 position with preferential coupling to the N1 site with a 7:5 isomer ratio (N1:N2). The amount of 1,2,4-triazolo-[4,3-a]pyrazines obtained can be dependent on the various reaction parameters, such as the concentration of tetrazole and pyrazine, type of electrolyte, anode and cathode material and charge and current used during the electrolysis process (see Table 1 and Table 2). Table 1 shows the reaction yield dependencies as a function of operating conditions for the electrochemical coupling of 5H-tetrazole with 2,6-dimethoxypyrazine.

TABLE 1

| | mmol | mmol | Electrolyte | | | | Charge | Current | 13a % | 12a % |
|---|---|---|---|---|---|---|---|---|---|---|
| Entry | Tetrazole | pyrazine | (200 mM) | Additive | Anode | Cathode | (F/mol) | (mA) | Yield | Yield |
| 1 | 2 | 2 | Et₄NClO4 | — | Pt | Pt | 2 | 5 | 15.4 | 22.6 |
| 2 | 2 | 2 | Et₄NClO4 | — | Pt | Pt | 2 | 7.5 | 16.1 | 19.7 |
| 3 | 2 | 2 | Et₄NClO4 | — | Pt | Pt | 2 | 10 | 12.3 | 19.2 |
| 4 | 2 | 2 | Et₄NClO4 | — | Pt | Pt | 2 | 15 | 14.9 | 15.9 |
| 5 | 2 | 2 | Et₄NClO4 | — | Pt | Pt | 2 | 20 | 12.0 | 29.3 |
| 6 | 2 | 2 | Et₄NClO4 | — | Pt | Pt | 2 | 25 | 7.7 | 13.0 |
| 7 | 2 | 2 | Et₄NClO4 | — | Pt | Pt | 2.1 | 7.5 | 18.8 | 26.7 |
| 8 | 2 | 2 | Et₄NClO4 | — | Pt | Pt | 2.2 | 7.5 | 15.9 | 25.2 |
| 9 | 2 | 2 | Et₄NClO4 | — | Pt | Pt | 2.3 | 7.5 | 17.1 | 25.2 |
| 10 | 2 | 2 | Et₄NClO4 | — | Pt | Pt | 2.4 | 7.5 | 15.6 | 23.3 |
| 11 | 2 | 2 | Et₄NClO4 | — | Pt | Pt | 3 | 7.5 | — | — |
| 12 | 2.5 | 2 | Et₄NClO4 | — | Pt | Pt | 2.1 | 7.5 | 10.6 | 15.6 |
| 13 | 1 | 1 | Et₄NClO4 | — | Pt | Pt | 2.1 | 7.5 | 16.8 | 22.6 |
| 14 | 3 | 3 | Et₄NClO4 | — | Pt | Pt | 2.1 | 7.5 | 17.2 | 25.3 |
| 15 | 4 | 4 | Et₄NClO4 | — | Pt | Pt | 2.1 | 7.5 | 18.3 | 21.3 |
| 16 | 5 | 5 | Et₄NClO4 | — | Pt | Pt | 2.1 | 7.5 | 16.0 | 28.3 |
| 17 | 2 | 2 | Et₄NClO4 | base | Pt | Pt | 2.1 | 7.5 | 17.1 | 27.2 |
| 18 | 2 | 2 | Et₄NClO4 | Acid | Pt | Pt | 2.1 | 7.5 | 17.3 | 27.6 |
| 19 | 2 | 2 | LiClO4 | — | Pt | Pt | 2.1 | 7.5 | — | — |
| 20 | 2 | 2 | Et₄NBF4 | — | Pt | Pt | 2.1 | 7.5 | 16.4 | 26.7 |
| 21 | 2 | 2 | Et₄NI | — | Pt | Pt | 2.1 | 7.5 | — | — |
| 22 | 2 | 2 | Et₄N 5H- | — | Pt | Pt | 2.1 | 7.5 | 21.4 | 29.6 |
| | | | tetrazolate | | | | | | | |
| 23 | 2 | 2 | Et₄NClO4 | — | G | Pt | 2.1 | 7.5 | 1.7 | 3.9 |
| 24 | 2 | 2 | Et₄NClO4 | — | Pt | Ni | 2.1 | 7.5 | 10.1 | 14.9 |
| | | | | | | Foam | | | | |
| 25 | 2 | 2 | Et₄NClO4 | — | Pt | S.S | 2.1 | 7.5 | 5.1 | 15.4 |

*S.S = Stainless Steel (Surface area = 8.16 cm²), G = Graphite (Surface area = 8.16 cm²), Pt = Platinum Foil (Surface area = 4 cm²), base = 1 eq. collidine, acid = 1 eq. acetic acid

Table 2 shows the reaction yield dependencies as a function of operating conditions for the electrochemical coupling of 5-bromo-tetrazole with 2,6-dimethoxypyrazine.

TABLE 2

| | | | | | | | | | N2 | N1 |
|---|---|---|---|---|---|---|---|---|---|---|
| | mmol | mmol | Electrolyte | | | | | Current | Couple % | Couple % |
| Entry | Tetrazole | pyrazine | 200 mM | Additive | Anode | Cathode | F/mol | (mA) | Yield | Yield |
| 1 | 2 | 2 | Et₄NClO₄ | — | Pt | Pt | 2.1 | 7.5 | 12.7 | 13.1 |
| 2 | 2 | 2 | Et₄NClO₄ | — | Pt | Pt | 2.1 | 15 | 38.8 | 17.7 |
| 3 | 2 | 2 | Et₄N 5- | — | Pt | Pt | 2.1 | 15 | 49.5 | 24.3 |
| | | | bromotetrazolate | | | | | | | |

The compounds obtained by the electrolysis process were analyzed using liquid chromatography-mass spectrometry (LC-MS), nuclear magnetic resonance spectroscopy (NMR, ¹H, ¹³C, ¹⁵N), infrared radiation (IR) spectroscopy, elemental analysis, differential scanning calorimetry/thermogravimetric analysis (DSC/TGA), UV Vis spectroscopy, and single crystal X-ray crystallography. Based on the results of all analysis, alongside the cyclic voltammetry studies, the potential mechanism of the electrolysis process can be: the reaction is initiated by the anodic oxidation of compound (10) to generate radical cation compound (16) which is prone to nucleophilic attack by a tetrazole molecule. This dehydrogenative coupling results in an isomeric mixture of two possible radicals in which C—N bond formation can occur at either the tetrazole N/site compound (17) or the tetrazole N2 site compound (18). Subsequent 1e-anodic oxidation of radicals (17) and (18) can yield the N1 coupled product (12a) and the N2 coupled product (13a), respectively (see Scheme 2).

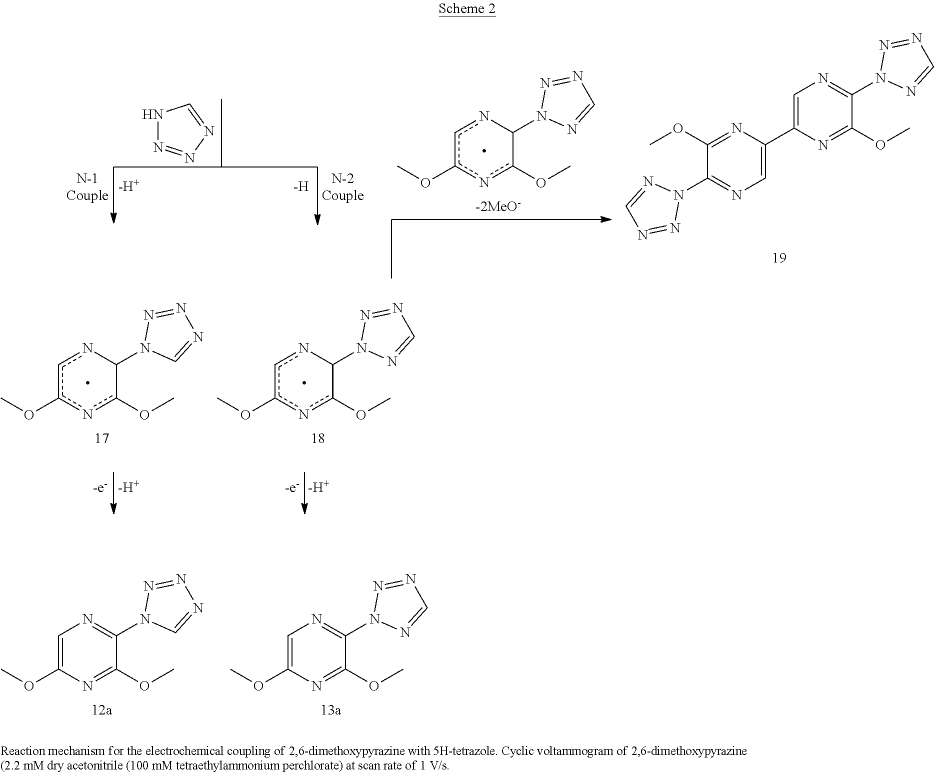

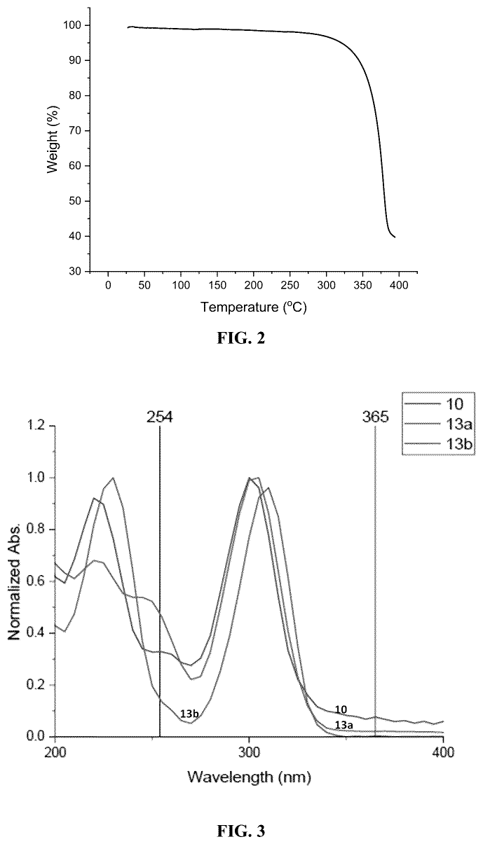

Further provided is a use of 3-substituted 1,2,4-triazolo-[4,3-a]pyrazine as a precursor in the synthesis of energetic materials. In some embodiments, the energetic material is 5-nitro-[1,2,4]triazolo[4,3-a]pyrazine-6,8-diamine (27). Scheme 3 illustrates the synthesis of a compound (27) from a compound (15a) by introducing a nitro and amino group, which can increase a thermal decomposition temperature by increasing inter and intramolecular hydrogen bonding. It is well-known that a high decomposition temperature is often necessary for the practical use of energetic materials. The energetic performance of compound (27) was evaluated (see Table 3) using the Gaussian09 and EXPLO5® software packages, indicating that 1,2,4-triazolo-[4,3-a]pyrazine can be employed as a precursor in the synthesis of energetic materials. The onset of decomposition was determined to be 294° C. by TGA (FIG. 2).

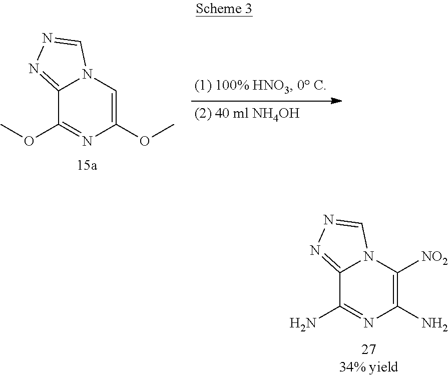

In some embodiments, 1,2,4-triazolo-[4,3-a]pyrazine of formula (IV) can be prepared using an electrolysis process alone comprising:
(i) reacting a compound of formula (I) with a compound of formula (II) in the presence of an organic solvent and an electrolyte to obtain a reaction mixture; and
(ii) subjecting the reaction mixture of step (i) to an electrolysis to obtain a cyclized product 1,2,4-triazolo-[4,3-a]pyrazine of formula (IV).

The compound of formulae (I), (II), (III) and (IV) are as defined above. The product can be obtained as a mixture of cyclized and uncyclized compounds of formulae (IV) and (III), respectively.

Any suitable organic solvent can be used in the electrolysis process. Examples of organic solvents used in the electrolysis process include, but are not limited to, acetone, ethyl acetate, dimethyl sulfoxide, N,N-dimethyl formamide, dichloromethane, acetonitrile, propionitrile, and tetrahydrofuran. Desirably, the organic solvent is acetonitrile. Any suitable electrolyte can be used. The electrolyte can be selected from tetraalkylammonium salts, trialkyl ammonium salts, tetraalkylammonium tetrazolate, organic or inorganic perchlorates, organic or inorganic tetrafluoroborates, organic or inorganic nitrates, and organic or inorganic hexafluorophosphates. In some embodiments, the electrolyte is selected from tetraethylammonium tetrafluoroborate, sodium tetrafluoroborate, sodium perchlorate, tetraethylammonium perchlorate, tetrapropylammonium perchlorate, tetraethylammonium tetrazolate, tetraethylammonium 5-bromo tetrazolate, tetrabutylammonium hexafluorophosphate and lithium nitrate.

In some embodiments, the electrolysis can be carried out using the current from about 5 mA to about 25 mA, such as from about 5 mA to 25 mA, 5 mA to about 25 mA, or 5 mA to 25 mA. Desirably, the electrolysis can be carried out using a current from about 7.5 mA to about 15 mA, such as from about 7.5 mA to 15 mA, 7.5 mA to about 15 mA, or 7.5 mA to 15 mA. Desirably, the electrolysis can be carried out using a current of about 7.5 mA (such as 7.5 mA).

In some embodiments, the optimal charge passed during the electrolysis can be from about 2 F/mol to about 3 F/mol, such as from about 2 F/mol to 3 F/mol, 2 F/mol to about 3 F/mol, or 2 F/mol to 3 F/mol. Desirably, the optimal charge passed during the electrolysis can be about 2.1 F/mol (such as 2.1 F/mol).

1,2,4-triazolo-[4,3-a]pyrazine of formula (IV) can also be prepared in a single-step. The method comprises performing an electrochemical process and a photochemical process simultaneously. The electrochemical coupling of a compound comprising 5-substituted tetrazole moiety to a compound comprising optionally substituted pyrazine moiety and the photochemical excitation of formed 2,5-disubstituted tetrazole using a UV light can be carried out simultaneously using the same conditions as described herein above.

The term “substituted” refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, and carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

Non-limiting examples of substituents, which can be bonded to a substituted carbon atom (or other atom, such as nitrogen) include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R wherein R can be hydrogen or a carbon-based moiety, and wherein the carbon-based moiety can itself be further substituted; for example, where R can be hydrogen, alkyl, acyl, cycloalkyl, aryl, aralkyl, heterocyclyl, heteroaryl, or heteroarylalkyl, any alkyl, acyl, cycloalkyl, aryl, aralkyl, heterocyclyl, heteroaryl, or heteroarylalkyl or R can be independently mono- or multi-substituted; or when two R groups bonded to a nitrogen atom or to adjacent nitrogen atoms can, together with the nitrogen atom or atoms to which they are bonded, form a heterocyclyl, the heterocycle can be mono- or independently multi-substituted.

The term “alkyl” refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₅) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

The term “alkynyl” refers to an unsaturated monovalent chain of carbon atoms, including at least one triple bond, which may be optionally branched. In various embodiments that include alkynyl, illustrative examples include lower alkynyl, such as C₂-C₆, C₂-C₄ alkynyl, and the like.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and cryloyl groups and the like. When the group containing the carbon atom that is bonded to the carbonyl carbon atom contains a halogen, the group is termed a “haloacyl” group. An example is a trifluoroacetyl group.

The term “aryl” refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” refers to substituted or unsubstituted aromatic and non-aromatic ring compounds containing three or more ring members, of which one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-ylpropyl.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein. The term “amino group” refers to a substituent of the form —NH₂, —NHR, —NR₂, —NR₃+, wherein each R is independently selected, and protonated forms of each, except for —NR₃+, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

The terms “halo,” “halogen,” and “halide” group, by themselves or as part of another substituent, mean, unless otherwise stated, a fluorine, chlorine, bromine, or iodine atom. The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl includetrifluoromethyl, 1,1-dichloroethyl, 1,2-dichloroethyl, 1,3-dibromo-3,3-difluoropropyl, perfluorobutyl, —CF(CH₃)₂ and the like.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialky-lamino, acylamino, thio, and the like, and combinations thereof.

The terms “optionally substituted” and “optional substituents” are used to describe groups, which are either unsubstituted or substituted with one or more of the substituents specified. When the groups in question are substituted with more than one substituent, the substituents can be the same or different. The terms “independently” “independently are” and “independently selected from” mean that the groups in question may be the same or different. Certain of the defined groups or substituents can occur more than once in the structure, and upon such occurrence each group or substituent shall be defined independently of the other.

The term “compound” as used herein, is meant to include all stereoisomers, geometric isomers, and tautomers of the structures depicted.

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

### EXAMPLES

### General Information

All reagents and solvents were used as received (Sigma-Aldrich, Fluka, Acros Organics, Fisher Scientific Co LLC). Melting and decomposition points were measured with a TA Instruments SDT Q600 TGA/DSC using heating rates of 5 K min⁻¹. ¹H, ¹³C, and ¹⁵N NMR spectra were measured using Bruker AV-III-500-HD (5 mm BBFO Cryoprobe Prodigy) Advance DRX NMR spectrometer. All chemical shifts are quoted in ppm relative to tetramethylsilane (¹H, ¹³C) or nitromethane (¹⁵N). Infrared spectra were measured using a Perkin-Elmer Spectrum two FT-IR spectrometer in the ATR mode. Transmittance values are described as “strong” (s), “medium” (m), and “weak” (w). Mass spectra were measured with an Agilent 1260 Infinity II Quaternary LC instrument using positive and negative ionizing modes. Elemental analysis was performed using a vario EL cube—Elemental Analyzer.

### Electrode Cleaning Procedure:

Platinum (Pt) foil electrodes were cleaned by direct heating with a propane blow torch. Once cool, the surfaces were rinsed with acetonitrile and dried before use. Graphite and stainless-steel electrodes were sonicated in acetone, water, and then acetonitrile sequentially.

### EXAMPLES

1H-tetrazole (11a) and 5-bromotetrazole (11b)

Compounds (11a) and (11b) were synthesized according to procedures well-known in the art (see, e.g., R. Bronisz, Inorganica Chim. Acta, 2002, 340, 215-220; D. R. Wozniak, et al., Zeitschrift für Anorg. und Allg. Chemie 2022, 648, e202100333, which are hereby specifically incorporated by reference for their teachings regarding same). ¹⁵N labeled tetrazole was synthesized by a modified version of this same procedure in which ¹⁵N labeled ammonium chloride and ¹⁵N labeled sodium azide were used.

### Tetraethylammonium Tetrazolate

Tetraethylammonium tetrazolate was prepared by combining 1 eq. of tetraethylammonium hydroxide with 1 eq. of 1H-tetrazole. The solvent was removed by rotary evaporation, and the sample was fully dried after storage in a desiccator with P₂O₅.

### Tetraethylammonium Bromtetrazolate

Tetraethylammonium bromtetrazolate was prepared by combining 1 eq. of tetraethylammonium hydroxide with 1 eq. bromotetrazole. The solvent was removed by rotary evaporation, and the sample was fully dried after storage in a desiccator with P₂O₅.

### 5-Azido-1H-tetrazole (11c)

5-Azido-1H-tetrazole was prepared from a modified procedure reorted by Klapötke et al, Journal of American Chemical Society, 2009, 131, 1122-1134, which is hereby specifically incorporated by reference. 5 mmol of cyanogen bromide was added to 20 mL of DI water at 0° C. To this solution, 10 mmol of NaN₃ dissolved in 5 mL of water was added dropwise while cooling in an ice bath. After stirring for 2 hours, 5 mL of 1 M HCl was added slowly, and the solution was allowed to warm to room temperature. The solution was then extracted 3×10 mL with cold diethyl ether. The combined organics were then dried over anhydrous magnesium sulfate.

### 5-Methoxy-1H-tetrazole (11d)

5-Methoxy-1H-tetrazole was prepared as a byproduct of the unmodified 5-azido-1H-tetrazole synthesis reported by Klapötke et al., Journal of American Chemical Society, 2009, 131, 1122-1134, which is hereby specifically incorporated by reference for its teachings regarding same.

Tetraethylammonium 5-phenyl-tetrazolate monohydrate (11h)

Tetraethylammonium 5-phenyl-tetrazolate monohydrate was prepared by combining 1 eq of tetraethylammonium hydroxide with 1 eq. 5-phenyl-1H-tetrazole. Solvent was removed by rotary evaporation, and the sample was fully dried after storage in a desiccator with P₂O₅. Calculated: 6.10% N, 41.83% C, 8.78% H, found 5.79% N, 40.91% C, 8.50% H (averaged for 3 replicates).

### 5-Cyclopropyl-1H-tetrazole (11i)

5-Cyclopropyl-1H-tetrazole was prepared using the procedure outlined by Sharpless et al., Journal of Organic Chemistry, 2001, 66, 7945-7950 in which cyclopropane carbonitrile was used as the nitrile source, which is hereby specifically incorporated by reference for its teachings regarding same.

### 5-(2,6-Difluorobenzyl)-1H-tetrazole (11j)

5-(2,6-Difluorobenzyl)-1H-tetrazole was prepared using the literature procedure disclosed by Sharpless et al., Journal of Organic Chemistry, 2001, 66, 7945-7950 in which 2,6-difluorobenzynitrile was used as the nitrile source, which is hereby specifically incorporated by reference for its teachings regarding same.

### 5-(3,3-Difluoroazetidin)-1H-tetrazole (11k)

5-(3,3-Difluoroazetidin)-1H-tetrazole was prepared using the reported procedure Piercey et al., Chemical Review, 2022, 122, 9, 8809-8840, which is hereby specifically incorporated by reference for its teachings regarding same.

### 5-(2-Nitrofuran)-1H-tetrazole (11l)

5-(2-Nitrofuran)-1H-tetrazole was prepared using the procedure outlined by Sharpless et al., Journal of Organic Chemistry, 2001, 66, 7945-7950 in which 5-nitrofuronitrile was used as the nitrile source, which is hereby specifically incorporated by reference for its teachings regarding same.

### 5,5′-Bistetrazole (11m)

5,5′-Bistetrazole was prepared using the literature procedure by Klapötke et al., Chemistry—A European Journal 2012, 18, 4051-4062, which is hereby specifically incorporated by reference for its teachings regarding same.

### 5-(Nitromethyl)-1H-tetrazole (11o)

5-(Nitromethyl)-1H-tetrazole was prepare using the literature procedure by Terpigorev et al., Russian Journal of Organic Chemistry, 1987, 21 (2), 244-252, which is hereby specifically incorporated by reference for its teachings regarding same.

Sodium 5-nitrotetrazolate (11p)

Sodium 5-nitrotetrazolate was prepared using the literature procedure by Klapötke et al., Anorg Allg Chem, 2013, 639, 681-688, which is hereby specifically incorporated by reference for its teachings regarding same.

### 5-Furan-1H-tetrazole (11q)

5-Furan-1H-tetrazole was prepared using the procedure outlined by Sharpless et al., Journal of Organic Chemistry, 2001, 66, 7945-7950, in which 5-nitrofuronitrile was used as the nitrile source, which is hereby specifically incorporated by reference for its teachings regarding same.

2-Chloro-6-methoxypyrazine (10c′)

2-Chloro-6-methoxypyrazine was prepared using the literature report (Pagoria et al., Synthesis, Scale-up and Characterization of 2,6-Diamino-3,5-Dinitropyrazine-1-Oxide (LLM-105), 1998), which is hereby specifically incorporated by reference for its teachings regarding same.

2,6-dimethoxy-3-[tetrazol-1-yl](12a)

2 mmol of 2,6-dimethoxy pyrazine, 2 mmol of 1H-tetrazole, and 2 mmol of tetraethylammonium tetrazolate were dissolved in 10 mL of dry acetonitrile and transferred to an Ika Electrasyn 2.0 sample vial. The septum of the sample vial was punctured with two needles to act as an inlet and outlet during oxygen purge. Argon gas was then bubbled through for 10 minutes to remove dissolved oxygen. Both needles were removed before electrolysis. 7.5 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 10 g HC DUO column. The solvent system used was a hexanes:ethyl acetate gradient. Yield=29.6%.

¹³C NMR (CD₃CN) S 160.4, 151.9, 143.4, 123.8, 123.1, 54.6, 54.5; ¹H NMR (CD₃CN) δ=9.29 (s, 1H); 7.79 (s, 1H); 4.06 (s, 6H); IR (cm⁻¹): 3403(w), 3146(w), 3109(w), 2959(w), 1654(w), 1587(w), 1544 (m), 1541(m), 1497(m), 1495(m), 1476(m), 1464(w), 1452(m), 1432(w), 1379(s), 1332(s), 1298(m), 1246(m), 1223(m), 1206(m), 1162(s), 1084(s), 1053(m), 1020 (w), 1001(m), 987(s), 955(m), 896(m), 882(s), 773(m), 725(s); MS (ESI+): m/z: 209.1 EA: (C₇H₈N₆O₂ 208.07 g/mol) calcd: 40.37% N, 40.38% C, 3.87% H, found 39.01% N, 38.79% C, 3.66% H (averaged for 6 replicates).

2,6-dimethoxy-3-[tetrazol-2-yl](13a)

The procedure for synthesizing compound (13a) is identical to compound (12a). Yield=21.4%.

¹³C {¹H} NMR (CD₃CN) δ=161.1, 153.3, 153.1, 125.6, 123.3, 54.6, 54.4; ¹H NMR (CD₃CN) δ 8.84 (s, 1H), 7.80 (s, 1H), 4.08 (s, 3H), 4.01 (s, 3H); IR (cm⁻¹): 3147(w), 2993(w), 2944(w), 2851(w), 1996(w), 1757(w), 1589 (m), 1552(s), 1487(m), 1471(s), 1457(m), 1431(m), 1390(m), 1371(s), 1336(s), 1282(m), 1255(m), 1212(m), 1200(s), 1183(s), 1157(m), 1125(s), 1086(m), 1038(m), 1019(m), 1002(s), 991(s), 893(m), 878(s), 786(w), 772(w), 729(m), 705(m), 688(s), 610(w), 572(m), 565(m), 511(s), 496(m), 465(w); MS (ESI+): m/z: 209.1: EA: (C₇H₈N₆O₂ 208.07 g/mol) calcd: 40.37% N, 40.38% C, 3.87% H, found 40.49% N, 40.56% C, 3.72% H (averaged for 6 replicates).

2,6-dimethoxy-3-(5-bromo-1H-tetrazol-1-yl)-pyrazine (12b)

2 mmol of 2,6-dimethoxy pyrazine, 2 mmol of bromotetrazole, and 2 mmol of tetraethylammonium bromotetrazolate were dissolved in 10 mL of dry acetonitrile and transferred to an Ika Electrasyn 2.0 sample vial. The septum of the sample vial was punctured with two needles to act as an inlet and outlet during oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed three times with water and once with brine. The organic layer was dried over anhydrous magnesium sulfate, filtered, and purified using a Biotage® Isolera™ flash chromatography system equipped with a 25 g HC DUO column. The solvent system used was a hexanes:ethyl acetate gradient. Yield=24.3%.

¹³C {¹H}NMR (dmso-d6) δ161.7, 154.0, 136.7, 125.1, 121.3, 54.8, 54.5; ¹H NMR (CD₃CN) δ 8.02 (s, 1H), 4.06 (s, 3H), 4.00 (s, 3H); IR (cm⁻¹): 2952(w), 1746(w), 1581(m), 1541(s), 1488(s), 1464(s), 1442 (m), 1424(w), 1416(m), 1396(w), 1373(s), 1348(s), 1329(s), 1283(w), 1250(m), 1239(m), 1194(s), 1176(s), 1105(s), 1064(w), 1049(w), 1017(s), 990(s), 970(m), 964(m), 920(w), 905(w), 874(m), 870(m), 777(m), 763(m), 732(s), 712(w), 700(w), 692(s), 622(w), 573(w), 561(w), 555(w), 532(w), 497(w), 471(w); MS (ESI+): m/z: 258.9, 261.0.

2,6-dimethoxy-3-(5-azido-tetrazol-1-yl)-pyrazine (12c)

2 mmol of 2,6-dimethoxy pyrazine, 2 mmol of compound (11c), and 2 mmol of tetraethylammonium perchlorate were dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=18.6%

¹³C{1H} NMR (CD₃CN) δ161.4, 153.9, 124.2, 123.2, 120.6, 54.7, 54.5; ¹H NMR (CD₃CN) δ 7.8(s, 1H), 4.06 (s, 3H), 4.00 (s, 3H); IR (cm⁻¹): 2954(w), 2156(s), 1693(w), 1589(m), 1551(s), 1516(s), 1487(s), 1475(s), 1451(s), 1418(m), 1379(s), 1334(s), 1315(m), 1296(m), 1248(m), 1209(m), 1189(s), 1171(s), 1096(m), 1078(m), 1067(m), 1029(m), 992(s), 957(s), 875(m), 803(w), 772(w), 725(m), 703(m), 599(w), 570(w), 530(w), 508(w), 496(m), 460(m); FTMS (ESI+): Expected m/z: 249.109999, Found m/z: 249.10979 (Error: −0.83 ppm).

2,6-Dimethoxy-3-(5-methoxy-tetrazol-1-yl)-pyrazine (12d)

2 mmol of 2,6-dimethoxy pyrazine was combined with a mixture of compound (11d) (0.4 mmol) and compound (11c) (1.4 mmol) and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=14.0%.

¹³C{1H}NMR (CD₃CN) δ162.2, 161.2, 154.1, 124.0, 121.1, 60.1, 54.6, 54.4; ¹H NMR (CD₃CN) δ7.80 (s, 1H), 4.19 (s, 3H), 4.05 (s, 3H), 3.99 (s, 3H); IR (cm⁻¹): 3004(w), 2953(w), 2853(w), 2757(w), 2642(w), 2145(w), 1709(m), 1622(m), 1600(m), 1552(m), 1485(m), 1460(s), 1429(m), 1396(m), 1332(m), 1275(m), 1195(s), 1130(m), 1056(m), 995(s), 966(m), 866(m), 740(m), 711(m), 691(m), 571(m), 536(m), 502(m), 414(m); FTMS (ESI+): Expected m/z: 239.089264, Found m/z: 239.08906 (Error: −0.85 ppm).

2,6-Dimethoxy-3-(5-ethylthio-tetrazol-1-yl)-pyrazine (12e)

2 mmol of 2,6-dimethoxy pyrazine was combined with 2 mmol of compound (11e) and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=3.5%

¹³C{1H}NMR (CD₃CN) δ161.7, 154.0, 136.7, 125.1, 121.3, 54.8, 54.5; ¹H NMR (CD₃CN) δ8.02 (s, 1H), 4.06 (s, 3H), 4.00 (s, 3H); IR (cm⁻¹): 2950(w), 1584(m), 1545(s), 1488(s), 1466(m), 1437(m), 1394(m), 1369(s), 1328(s), 1263(m), 1238(m), 1177(s), 1084(m), 1060(m), 1022(m), 996(s), 969(m), 869(m), 777(w), 763(w), 731(m), 693(m), 565(m), 509(w), 487(w), 466(m); FTMS (ESI+): Expected m/z: 269.082071, Found m/z: 269.08221 (Error: 0.51 ppm).

2,6-Dimethoxy-3-(5-methyl-tetrazol-1-yl)-pyrazine (12f)

2 mmol of 2,6-dimethoxy pyrazine was combined with 2 mmol of compound (11f) and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=16.2%

¹³C{1H}NMR (CD₃CN) δ161.1, 153.7, 153.6, 123.9, 122.6, 54.6, 54.4, 8.2; ¹H NMR (CD₃CN) δ7.80 (s, 1H), 4.06 (s, 3H), 4.00 (s, 3H), 2.44 (s, 3H); IR (cm⁻¹): 2953(w), 1731(w), 1551(m), 1519(m), 1457(m), 1361(s), 1299(m), 1238(s), 1203(m), 1129(m), 1110(m), 1080(m), 1040(m), 998(s), 913(m), 854(m), 788(w), 712(w), 682(m), 606(m), 560(m), 517(m), 430(m); FTMS (ESI+): Expected m/z: 223.094349, Found m/z: 223.09401(Error: −1.51 ppm).

2,6-Dimethoxy-3-(5-isobutyl-tetrazol-2-yl)-pyrazine (12g)

2 mmol of 2,6-dimethoxy pyrazine was combined with 2 mmol of 5-aminotetrazole and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=15.3%

¹³C{1H}NMR (CD₃CN) δ161.2, 156.1, 154.0, 124.0, 122.6, 54.6, 54.4, 31.7, 27.2, 21.4; ¹H NMR (CD3CN) S 7.81 (s, 1H), 4.07 (s, 3H), 3.98 (s, 3H), 2.25 (d, 2H), 2.04 (septet, 1H), 0.89 (d, 6H); IR (cm⁻¹): 2958(m), 2873(w), 1585(m), 1545(s), 1489(s), 1467(s), 1378(s), 1328(s), 1268(m), 1238(m), 1181(s), 1110(m), 1080(m), 1027(m), 997(s), 869(m), 825(w), 777(m), 730(m), 712(m), 700(m), 684(m), 607(w), 574(m), 560(m), 526(m), 499(m), 465(m), 414(w) FTMS (ESI+): Expected m/z: 265.141299, Found m/z: 265.14113(Error: −0.63 ppm).

2,6-Dimethoxy-3-(5-phenyl-tetrazol-1-yl)-pyrazine (12h):

2 mmol of 2,6-dimethoxy pyrazine was combined with 2 mmol of compound (11h) and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=3.0%

¹³C{1H}NMR (CD₃CN) δ161.4, 155.0, 153.8, 130.8, 131.5, 129.2, 128.3, 126.8, 124.3, 123.6, 123.1, 54.7, 54.3; ¹H NMR (CD₃CN) δ7.83 (s, 1H), 7.56(q, 3H), 7.46 (t, 2H) 4.05 (s, 3H), 3.82 (s, 3H); IR (cm⁻¹): 2950(w), 1586(m), 1544(s), 1488(s), 1464(s), 1430(m), 1402(w), 1373(s), 1330(s), 1276(m), 1238(m), 1185(s), 1131(m), 1097(m), 1073(m), 1028(m), 996(s), 925(w), 870(m), 777(m), 726(m), 688(s), 618(w), 552(m), 509(m), 493(m), 470(m), 421(w); FTMS (ESI+): Expected m/z: 285.11000, Found m/z: 285.10984(Error: −0.561 ppm).

2,6-Dimethoxy-3-(5-cycloproyl-tetrazol-1-yl)-pyrazine (12i):

2 mmol of 2,6-dimethoxy pyrazine was combined with 2 mmol of 5-cyclopropyltetrazole (2 mmol) and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=5.4%

¹³C{1H}NMR (CD₃CN) δ161.1, 159.0, 154.0, 124.0, 123.6, 54.6, 54.4, 8.86, 3.88; ¹H NMR (CD₃CN) δ7.83 (s, 1H), 4.08 (s, 3H), 4.01 (s, 3H), 1.87(pentet, 1H), 1.43 (s, 4H); IR (cm⁻¹): 3009(w), 2951(w), 1765(w), 1726(w), 1585(m), 1545(m), 1524(m), 1489(s), 1468(m), 1455(m), 1386(s), 1358(s), 1327(s), 1258(m), 1237(m), 1176(s), 1094(m), 1065(m), 1049(m), 1023(m), 997(s), 871(m), 820(m), 779(m), 745(m), 732(m), 703(m), 653(m), 560(m), 480(m); FTMS (ESI+): Expected m/z: 249.109999, Found m/z: 249.10979(Error: −0.83 ppm).

2,6-Dimethoxy-3-(5-(2,6-difluorobenzyl)-tetrazol-1-yl)-pyrazine (12j):

2 mmol of 2,6-dimethoxy pyrazine was combined with 2 mmol of 5-(2,6-difluorobenzy)-tetrazole (2 mmol) and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=15.4%

¹³C{1H}NMR (CD₃CN) δ162.04(d, J=0.07), 161.20, 160.4(d, J=0.06), 160.06(d, J=0.06), 154.28, 153.77, 130.17(q, J=0.08), 111.39(d, J=0.20), 111.34(d, J=0.19), 110.32(t, J=0.16); 54.64, 54.31, 17.05(t, J=0.03); ¹H NMR (CD₃CN) 7.73 (s, 1H), 7.31 (m, 1H), 6.91 (t, 2H), 4.28 (s, 2H), 4.04 (s, 3H), 3.90 (s, 3H); IR (cm⁻¹): 2954(w), 1736(w), 1627(m), 1592(m), 1546(m), 1490(m), 1469(m), 1426(m), 1380(m), 1338(m), 1297(m), 1270(m), 1235(m), 1185(s), 1143(m), 1104(m), 1073(m), 1057(m), 1043(m), 1012(s), 878(m), 838(m), 798(m), 774(s), 733(m), 699(m), 661(m), 547(m), 535(m), 493(m), 453(m); FTMS (ESI+): Expected m/z: 335.106805, Found m/z: 335.10683(Error: 0.07 ppm).

2,6-Dimethoxy-3-(5-(3,3-difluoroazetidin)-tetrazol-1-yl)-pyrazine (12k):

2 mmol of 2,6-dimethoxy pyrazine was combined with 2 mmol of 5-(3,3-difluoroazetidin)-1H-tetrazole and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=2.3%

¹³C{1H}NMR (CD₃CN) δ161.7, 161.3 156.4, 154.0, 153.2, 146.1, 144.1, 140.0, 125.2, 124.2, 123.6, 122.1, 114.3, 113.4, 133.31 112.7, 54.8, 54.7, 54.6, 54.5; ¹H NMR (CD₃CN) δ7.79 (s, 1H), 4.32 (t, 4H), 4.06 (s, 3H), 4.02 (s, 3H); IR (cm⁻¹): 2954(w), 1672(w), 1547(s), 1489(m), 1455(m), 1381(m), 1352(s), 1333(m), 1242(s), 1226(s), 1185(s), 1093(m), 1051(m), 999(m), 910(m), 778(m), 755(m), 732(m), 715(m), 530(m), 508(m), 474(m), 420(m); FTMS (ESI+): Expected m/z: 300.102053, Found m/z: 300.10206(Error: 0.02 ppm).

2,6-Dimethoxy-3-(5-nitrofuran-2-yl)-tetrazol-1-yl)-pyrazine (12l) and 2,6-dimethoxy-3-(5-nitrofuran-2-yl)-tetrazol-2-yl)-pyrazine (13l)

2 mmol of 2,6-dimethoxy pyrazine was combined with 2 mmol of 5-(2-nitrofuran)-tetrazole and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=6%

¹³C{1H}NMR (CD₃CN) δ161.7, 161.3 156.4, 154.0, 153.2, 146.1, 144.1, 140.0, 125.2, 124.2, 123.6, 122.1, 114.3, 113.4, 133.31 112.7, 54.8, 54.7, 54.6, 54.5; ¹H NMR (CD₃CN) δ7.87(s, 0.7H), 7.83 (s, 1H), 7.61 (d, 1H), 7.48(d, 0.7H), 7.46 (d, 1H), 7.19(d, 0.7H), 4.11(s, 2.1H), 4.10 (s, 3H), 4.06 (s, 3H), 3.93(s, 2.1H); IR (cm⁻¹): 3118(w), 1587(w), 1549.59, 1513(m), 1484(m), 1458(m), 1421(w), 1409(w), 1386(m), 1351(s), 1331(s), 1244(m), 1192(s), 1139(m), 1094(m), 1021(m), 991(s), 968(m), 918(m), 864(m), 827(m), 811(s), 751(m), 737(m), 728(m), 703(m), 653(w), 569(m), 531(w), 503(m), 485(m), 428(w); FTMS (ESI+): Expected m/z: 320.07434, Found m/z: 320.07441(Error: 0.219 ppm).

2,6-Dimethoxy-3-(5-amino-tetrazol-1-yl)-pyrazine (12n):

The procedure for compound (12n) is identical to the procedure for compound (12c). Yield=3%

¹³C{1H}NMR (dmso-d6) δ160.6, 156.4, 154.5, 123.9, 122.7, 55.1, 54.8; ¹H NMR (CD₃CN) δ 7.90 (s, 1H), 6.88 (s, 2H), 4.02 (s, 3H), 3.95 (s, 3H); IR (cm⁻¹): 3362(w), 3133(w), 2953(w), 2147(w), 1656(m), 1583(m), 1545(m), 1490(m), 1452(m), 1422(m), 1379(m), 1337(m), 1316(m), 1233(m), 1178(s), 1124(m), 1100(m), 1067(m), 1044(m), 993(s), 880(m), 765(m), 737(m), 723(m), 701(m), 685(m), 623(m), 586(m), 574(m), 513(m), 486(m), 410(m); FTMS (ESI+): Expected m/z: 224.0889598, Found m/z: 224.08993(Error: 1.48 ppm).

2-(5-Bromo-tetrazol-1-yl)-5-chloro-3-methoxypyrazine (12c′):

5 mmol of 2-methoxy-6-chloro-pyrazine was combined with 5 mmol of 5-bromotetrazole and 5 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=0.8%

¹³C{1H}NMR (CD₃CN) δ153.3, 147.9, 143.5, 134.0, 132.1, 55.6; ¹H NMR (CD₃CN) δ8.28 (s, 1H), 4.06 (s, 3H), IR (cm⁻¹): 1573(w), 1529(w), 1466(m), 1431(m), 1376(s), 1253(m), 1138(s), 1071(m), 1028(s), 981(s), 917(m), 715(m), 639(m), 568(m), 499(m); FTMS (ESI+): Expected m/z: 262.933525(M−2N), Found m/z: 262.93290(M−2N) (Error: −2.37 ppm).

2,6-dimethoxy-3-(5-bromo-1H-tetrazol-2-yl)-pyrazine (13b)

The procedure for synthesizing compound (13b) is identical to compound (12b). Yield=49.5%.

¹³C {¹H} NMR (CD₃CN) δ161.3, 153.2, 143, 125.0, 123.5, 54.8, 54.5; ¹H NMR (CD₃CN) δ=7.77 (s, 1H), 4.06 (s, 3H), 4.00 (s, 3H); IR (cm⁻¹): 2944(w), 1738(w), 1567(w), 1533(w), 1475(w), 1447(w), 1410(w), 1373(s), 1320(s), 1310(s), 1251(w), 1228(w), 1182(m), 1160(m), 1101(w), 1079(m), 1039(s), 993(s), 950(w), 941(w), 868(m), 755(w), 734(w), 716(s), 688(s), 570(w), 517(w), 487(s), 458(w); MS (ESI+): m/z: 258.9, 261.0; EA: (C₇H₇BrN₆O₂ 285.98 g/mol) calcd: 29.28% N, 29.29% C, 2.46% H, found 29.10% N, 28.92% C, 2.33% H (averaged for 3 replicates).

2,6-Dimethoxy-3-(5-azido-tetrazol-2-yl)-pyrazine (13c)

The procedure for synthesizing compound (13c) is identical to compound (12c). Yield=27.2%

¹³C{1H}NMR (CD₃CN) δ162.4, 161.1, 153.2, 125.3, 123.4, 54.7, 54.5; ¹H NMR (CD₃CN) δ7.76 (s, 1H), 4.06 (s, 3H), 4.02 (s, 3H); IR (cm⁻¹): 2951(w), 2418(w), 2138(s), 1586(m), 1547(m), 1500(s), 1488(s), 1462(m), 1429(m), 1395(m), 1363(s), 1326(s), 1253(m), 1220(m), 1187(s), 1103(m), 1054(w), 1031(m), 994(s), 870(m), 804(w), 788(m), 776(m), 735(m), 701(m), 689(m), 571(m), 531(m), 491(w), 469(m); FTMS (ESI+): Expected m/z: 250.08009, Found m/z: 250.07999(Error: 0.399 ppm).

2,6-Dimethoxy-3-(5-methoxy-tetrazol-2-yl)-pyrazine (13d)

The procedure for synthesizing compound (13d) is identical to compound (12d). Yield=12.3%

¹³C{1H}NMR (CD₃CN) δ172.7, 160.9, 153.3, 125.8, 121.3, 58.3, 54.6, 54.4; ¹H NMR (CD₃CN) δ7.77 (s, 1H), 4.13 (s, 3H), 4.06 (s, 3H), 4.02 (s, 3H); IR (cm⁻¹): 2958(w), 2161(w), 1558(s), 1546(s), 1490(s), 1466(m), 1435(m), 1421(m), 1399(m), 1387(m), 1363(s), 1341(s), 1240(m), 1184(m), 1109(m), 1065(m), 1031(m), 1016(m), 980(s), 952(m), 865(m), 779(w), 753(m), 714(m), 696(m); FTMS (ESI+): Expected m/z: 239.089264, Found m/z: 239.08895(Error: −1.31 ppm).

2,6-Dimethoxy-3-(5-ethylthio-tetrazol-2-yl)-pyrazine (13e)

The procedure for synthesizing compound (13e) is identical to compound (12e). Yield=5.0%

¹³C{1H}NMR (CD₃CN) δ164.4, 161.0, 153.2, 125.5, 123.3, 54.7, 54.5, 26.3, 14.3; ¹H NMR (CD₃CN) δ7.78 (s, 1H), 4.07 (s, 3H), 4.02 (s, 3H). 3.27 (t, 3H), 3.27 (q, 3H), 1.42 (t, 3H); IR (cm⁻¹): 2949(w), 1583(m), 1545(s), 1488(s), 1465(m), 1437(m), 1394(m), 1368(s), 1328(s), 1263(m), 1238(m), 1177(s), 1084(m), 1059(m), 1022(m), 996(s), 969(m), 869(m), 777(m), 763(w), 731(m), 693(m), 565(m), 509(w), 487(m), 466(m); FTMS (ESI+): Expected m/z: 269.08207, Found m/z: 269.08188(Error: 0.603 ppm).

2,6-Dimethoxy-3-(5-methyl-tetrazol-2-yl)-pyrazine (13f)

The procedure for synthesizing compound (13f) is identical to compound (12f). Yield=16.2%

¹³C{1H}NMR (CD₃CN) δ163.1, 160.9, 153.2, 125.8, 123.2, 54.6, 54.4, 10.0 ¹H NMR (CD₃CN) δ7.76 (s, 1H), 4.06 (s, 3H), 4.00 (s, 3H), 2.59 (s, 3H); IR (cm⁻¹): 3005(w), 2952(w), 1587(m), 1544(m), 1513(m), 1483(m), 1462(s), 1432(m), 1394(m), 1362(m), 1346(m), 1329(s), 1254(m), 1209(m), 1198(m), 1171(s), 1096(m), 1032(m), 997(s), 961(m), 888(m), 875(m), 774(w), 740(s), 713(m), 707(m), 679(m), 570(m), 558(m), 517(m), 492(w), 454(m); FTMS (ESI+): Expected m/z: 223.09434, Found m/z: 223.094(Error: 1.524 ppm).

2,6-Dimethoxy-3-(5-isobutyl-tetrazol-2-yl)-pyrazine (13g)

The procedure for synthesizing compound (13g) is identical to compound (12g). Yield=9.7%

¹³C{1H}NMR (CD₃CN) δ166.0, 160.9, 153.3, 125.9, 123.2, 54.6, 54.4, 33.7, 27.8, 21.5; ¹H NMR (CD₃CN) δ7.77 (s, 1H), 4.06 (s, 3H), 4.00 (s, 3H), 2.85 (d, 2H), 2.16(septet, 1H), 0.99 (d, 6H); IR (cm⁻¹): 2956(m), 2872(w), 1586(m), 1547(m), 1497(m), 1488(m), 1463(m), 1430(m), 1394(m), 1363(s), 1327(s), 1231(m), 1185(s), 1139(m), 1105(m), 1062(w), 1032(m), 995(s), 961(m), 868(m), 778(w), 735(m), 697(m), 572(m), 522(m), 465(m); FTMS (ESI+): Expected m/z: 265.14129, Found m/z: 265.14113(Error: 0.603 ppm).

2,6-Dimethoxy-3-(5-phenyl-tetrazol-2-yl)-pyrazine (13h)

The procedure for synthesizing compound (13h) is identical to compound (12h). Yield=3.5%

¹³C{1H}NMR (CD₃CN) δ164.9, 161.0, 153.3, 130.8, 129.2, 127.1, 126.8, 125.7, 123.4, 54.7, 54.5; 1H NMR (CD₃CN) δ8.20 (multiplet, 2H), 7.82 (s, 1H), 7.57(multiplet, 3H) 4.08 (s, 3H), 4.03 (s, 3H); IR (cm⁻¹): 2950(w), 1586(m), 1544(s), 1488(s), 1464(s), 1430(m), 1402(w), 1373(s), 1330(s), 1276(m), 1238(m), 1185(s), 1131(m), 1097(m), 1073(m), 1028(m), 996(s), 925(w), 870(m), 777(m), 726(m), 688(m), 618(w), 552(m), 509(m), 493(m), 470(m), 421(w); FTMS (ESI+): Expected m/z: 285.109999, Found m/z: 285.11006(Error: 0.21 ppm).

2,6-Dimethoxy-3-(5-cycloproyl-tetrazol-2-yl)-pyrazine (13i)

The procedure for synthesizing compound (13i) is identical to compound (12i). Yield=3.0%

¹³C{1H}NMR (CD₃CN) δ168.7, 160.9, 153.3, 125.8, 123.2, 54.6, 54.4, 8.0, 6.1; 1H NMR (CD₃CN) δ7.77 (s, 1H), 4.07 (s, 3H), 4.01 (s, 3H), 2.28(septet, 1H), 1.15(multiplet, 2H), 1.07(multiplet, 2H); IR (cm⁻¹): 3007(w), 2950(w), 1738(w), 1586(m), 1546(s), 1520(m), 1488(s), 1463(m), 1430(m), 1394(m), 1362(s), 1327(s), 1275(m), 1244(m), 1185(s), 1105(m), 1082(m), 1030(m), 994(s), 869(m), 820(m), 778(m), 756(w), 735(m), 701(m), 647(w), 572(m), 521(m), 499(m), 465(m); FTMS (ESI+): Expected m/z: 249.10999, Found m/z: 249.10988(Error: 0.441 ppm).

2,6-Dimethoxy-3-(5-(2,6-difluorobenzyl)-tetrazol-2-yl)-pyrazine (13j)

The procedure for synthesizing compound (13j) is identical to compound (12j). Yield=9.7%

¹³C{1H}NMR (CD₃CN) δ164.2, 162.4(d, J=0.07), 160.9, 160.4(d, J=0.06), 153.2, 129.7(t, J=0.09), 125.6, 123.3, 122.5(t, J=0.16 ppm), 111.5(d, J=0.2 ppm), 111.4(d, J=0.12), 54.6, 54.4, 18.7(t, J=0.03); ¹H NMR (CD₃CN) δ7.75 (s, 1H), 7.37 (m, 1H), 7.03 (t, 2H), 4.40 (s, 2H), 4.05 (s, 3H), 3.99 (s, 3H); IR (cm⁻¹): 2954(w), 1736(w), 1627(m), 1592(m), 1546(m), 1490(m), 1469(s), 1426(m), 1380(m), 1338(m), 1297(m), 1270(m), 1235(m), 1185(s), 1143(w), 1104(m), 1073(m), 1057(m), 1043(m), 1012(s), 878(m), 838(m), 798(m), 774(s), 733(m), 699(m), 661(m), 547(m), 535(m), 493(m), 453(w); FTMS (ESI+): Expected m/z: 335.106805, Found m/z: 335.10686(Error: 0.16 ppm).

2,6-Dimethoxy-3-(5-(3,3-difluoroazetidin)-tetrazol-2-yl)-pyrazine (13k)

The procedure for synthesizing compound (13k) is identical to compound (12k). Yield=1.8%

¹³C{1H}NMR (CD₃CN) δ169.3, 160.8, 153.3, 125.8, 123.2, 64.1, 63.8(t, J=0.21), 54.6, 54.4; ¹H NMR (CD₃CN) δ7.76 (s, 1H), 4.54 (s, 1H), 4.52 (s, 2H), 4.49 (s, 1H), 4.06 (s, 3H), 4.02 (s, 3H); IR (cm⁻¹): 2952(w), 1722(w), 1548(s), 1489(m), 1462(m), 1430(m), 1397(m), 1365(s), 1351(s), 1329(s), 1242(s), 1184(s), 1105(m), 1057(m), 1035(m), 1000(m), 983(m), 911(m), 891(m), 798(w), 756(m), 735(m), 715(m), 571(w), 493(m), 473(m), 420(m); FTMS (ESI+): Expected m/z: 300.102053, Found m/z: 300.10188(Error: −0.57 ppm).

3-(5-Bromo-tetrazol-2-yl)-N,N-dimethylpyrazin-2-amine (13a′):

2 mmol of N,N-dimethylpyrazin-2-amine was combined with 2 mmol of 5-bromotetrazole and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=3.3%

¹³C{1H}NMR (CD₃CN) δ151.1, 145.4, 143.4, 137.7, 130.9, 38.4, 34.6; ¹H NMR (CD₃CN) δ8.38 (d, 1H), 7.88 (d, 1H), 2.72 (s, 6H); IR (cm⁻¹): 3056(w), 1680(m), 1648(m), 1579(s), 1510(m), 1464(m), 1407(s), 1351(m), 1320(m), 1285(s), 1256(m), 1187(m), 1165(m), 1094(m), 1054(m), 1024(s), 994(m), 957(w), 847(w), 815(w), 715(m), 595(m), 560(m), 526(m), 430(m); FTMS (ESI+): Expected m/z: 242.00413(M−2N), Found m/z: 242.00378(M−2N) (Error: 1.44 ppm).

2-Methoxy-3-(5-bromo-1H-tetrazol-2-yl)-pyrazine (13b′)

2 mmol of 2-methoxypyrazine was combined with 2 mmol of 5-bromotetrazole and 2 mmol of tetraethylammonium perchlorate and then dissolved in 10 mL of dry acetonitrile and transferred to an Ika Electrasyn 2.0 sample vial. The septum of the sample vial was punctured with two needles to act as an inlet and outlet during the oxygen purge. Argon gas was then bubbled through for 10 minutes to remove dissolved oxygen. Both needles were removed before electrolysis. 15 mA constant current electrolysis was then performed until 2.1 F/mol of charge had passed. Once electrolysis was complete, the acetonitrile was removed by rotary evaporation. The sample was then redissolved in ethyl acetate and washed 3 times with water and once with brine. The organic layer was then dried over anhydrous magnesium sulfate, filtered, and purified using a Biotage® Isolera™ flash chromatography system equipped with a 25 g HC DUO column. The solvent system used was a hexanes:ethyl acetate gradient. Yield=3.9%

¹³C{1H}NMR (CD₃CN) δ154.3, 145.1, 143.3, 135.3, 133.7, 54.7; ¹H NMR (CD₃CN) δ8.51 (d, 1H), 8.27 (d, 1H), 4.05 (s, 3H); IR (cm⁻¹): 2926(w), 1692(w), 1583(m), 1535(m), 1473(m), 1450(m), 1427(m), 1412(s), 1393(m), 1352(s), 1305(m), 1287(s), 1228(m), 1185(m), 1167(m), 1115(m), 1098(m), 1063(m), 1028(s), 1011(m), 993(s), 868(m), 855(m), 781(w), 715(m), 703(m), 673(w), 605(m), 578(w), 545(m), 511(m), 430(w); FTMS (ESI+): Expected m/z: 228.97249(M−2N), Found m/z: 228.9718(M−2N) (Error: 3.01 ppm).

6,8-dimethoxy-1,2,4-triazolo-[4,3-a]pyrazine (15a)

100 mg of compound (13a) was dissolved into 20 mL of acetonitrile in a 100 mL quartz test tube. The solution was then irradiated for 48 hours using 254 nm UV light in a dark room. Yield determined by quantitative NMR to be 65.3%. The sample was collected by slow evaporation of acetonitrile yielding a light white powder. Solids washed with minimal cold acetonitrile.

¹³C{¹H}NMR (dmso-d6) δ=152.1, 151.4, 139.0, 138.1, 93.0, 54.8, 54.6; ¹H NMR (dmso-d6) δ=9.24 (s, 1H), 4.07 (s, 3H), 3.80 (s, 3H); IR (cm-1): 3133(w), 3108(w), 3013(w), 2957(w), 1721(m), 1683(w), 1631(s), 1567(m), 1548(m), 1502(s), 1465(m), 1452(m), 1432(m), 1423(s), 1394(m), 1372(s), 1351(s), 1304(s), 1269(w), 1236(s), 1207(s), 1196(s), 1186(s), 1161(m), 1065(s), 1021(m), 980(s), 90(s), 942(s), 880(w), 840(s), 801(w), 778(s), 748(m), 704(w), 674(w), 661(m), 649(m), 628(s), 575(s), 531(w), 499(w); MS (ESI+): m/z: 181.1: EA: (C₇H₈N₄O₂ 180.06 g/mol) calcd: 31.10% N, 46.67% C, 4.48% H, found 31.23% N, 43.42% C, 4.33% H (averaged for 6 replicates).

3-bromo-6,8-dimethoxy-1,2,4-triazolo-[4,3-a]pyrazine (15b)

100 mg of compound (13a) was dissolved into 20 mL of acetonitrile in a 100 mL quartz test tube. The solution was then irradiated for 48 hours using 254 nm UV light in a dark room. Yield determined by quantitative NMR to be 51.3%. Sample collected by slow evaporation of acetonitrile yielding a light white powder. Solids washed with minimal cold acetonitrile.

¹³C{¹H}NMR (dmso-d6) δ153.1, 151.6, 140.0, 123.0, 91.7, 56.3, 55.3; ¹H NMR (dmso-d6) δ 7.36 (s, 1H), 4.09 (s, 3H), 3.87 (s, 3H); IR (cm⁻¹): 2963(w), 1686(w), 1625(w), 1557(w), 1493(w), 1447(w), 1424(w), 1375(m), 1338(w), 1305(w), 1246(m), 1201(m), 1171(m), 1067(w), 1048(w), 1013(s), 942(m), 848(m), 723(s), 630(s), 502(w); m/z: 258.9, 261.0; EA: (C₇H₇BrN₄O₂ 257.98 g/mol) calcd: 21.63% N, 32.5% C, 2.72% H, found 21.58% N, 31.60% C, 2.64% H (averaged for 2 replicates).

3-Azido-6,8-dimethoxy-1,2,4-triazolo-[4,3-a]pyrazine (15c)

43 mg of compound (13c) was dissolved into 1 mL of acetonitrile-d3 in a borosilicate NMR tube. The solution was then irradiated for 48 hours using 254 nm UV light in a dark room. Yield determined by quantitative NMR to be 32.6%. Sample purified using a Biotage® Isolera™ flash chromatography system equipped with a 25 g HC DUO column. The solvent system used was a ethyl acetate:acetonitrile gradient.

¹³C{1H} NMR (dmso-d6) δ152.4, 151.5, 143.3, 137.7, 90.5, 56.2, 55.1; ¹H NMR (dmso-d6) δ 7.15 (s, 1H), 4.07 (s, 3H), 3.80 (s, 3H); IR (cm⁻¹) 3201(w), 2924(w), 2853(w), 2151(m), 1681(m), 1633(m), 1550(m), 1501(s), 1458(m), 1382(m), 1333(m), 1290(m), 1269(m), 1212(s), 1049(m), 996(m), 629(m), 506(m); FTMS (ESI+): Expected m/z: 222.073948, Found m/z: 222.07352(Error: −1.92 ppm).

3-Methoxy-6,8-dimethoxy-1,2,4-triazolo-[4,3-a]pyrazine (15d)

44 mg of compound (13d) was dissolved into 1 mL of acetonitrile-d3 in a borosilicate NMR tube. The solution was then irradiated for 48 hours using 254 nm UV light in a dark room. Yield determined by quantitative NMR to be 21.8%. The dried sample was washed with ethyl acetate to yield pure slightly yellow solid.

¹³C{1H}NMR (CD₃CN) δ154.9, 151.8, 151.6, 136.4, 88.8, 58.3, 55.4, 54.2; ¹H NMR (CD₃CN) δ6.89 (s, 1H), 4.27 (s, 3H), 4.10 (s, 3H), 3.82 (s, 3H); IR (cm⁻¹): 2944 (w), 1625 (m), 1575 (m), 1550 (s), 1495 (s), 1481 (m), 1458 (m), 1449 (m), 1419 (m), 1378 (m), 1366 (m), 1329 (m), 1286 (m), 1199 (s), 1185 (m), 1170 (m), 1087 (s), 1061(m), 1035 (s), 989 (w), 943 (m), 862 (w), 754 (w), 712 (m), 691 (m), 655 (w), 624 (s), 561 (w), 503 (w), 439 (m); FTMS (ESI+): Expected m/z: 211.083116, Found m/z: 211.08268(Error: −2.06 ppm).

3-Ethylthio-6,8-dimethoxy-1,2,4-triazolo-[4,3-a]pyrazine (15e)

17 mg of compound (13e) was dissolved into 1 mL of acetonitrile-d3 in a borosilicate NMR tube. The solution was then irradiated for 48 hours using 254 nm UV light in a dark room. Yield determined by quantitative NMR to be 70.8%. Sample purified using a Biotage® Isolera™ flash chromatography system equipped with a 25 g HC DUO column. The solvent system used was a hexanes:ethyl acetate gradient.

¹³C{1H}NMR (CD₃CN) δ152.9, 152.0, 143.2, 139.8, 91.3, 55.5, 54.5; 28.5, 14.7; ¹H NMR (CD₃CN) δ7.24 (s, 1H), 4.15 (s, 3H), 3.90 (s, 3H), 3.10 (q, 2H), 1.30 (t, 3H); IR (cm⁻¹): 3134 (w), 3028 (w), 2969 (w), 2953 (w), 2926 (w), 2864 (w), 1726 (w), 1621 (s), 1555 (s), 1488 (s), 1460 (s), 1440 (m), 1429 (m), 1416 (s), 1398 (m), 1372 (s), 1322 (s), 1294 (s), 1250 (s), 1234 (s), 1201 (s), 1179 (s), 1166 (s), 1069 (m), 1056 (s), 1024 (s), 977 (m), 945 (s), 849 (s), 795 (m), 767 (m), 738 (s), 716 (m), 697 (m), 660 (w), 634 (s), 605 (w), 583 (s), 502 (m), 486 (m), 430 (m), 403 (w); FTMS (ESI+): Expected m/z: 241.075923, Found m/z: 241.07573(Error: −0.80 ppm).

3-Methyl-6,8-dimethoxy-1,2,4-triazolo-[4,3-a]pyrazine (15f)

52 mg of compound (13f) was dissolved into 1 mL of acetonitrile-d3 in a borosilicate NMR tube. The solution was then irradiated for 48 hours using 254 nm UV light in a dark room. Yield determined by quantitative NMR to be 56.2%. Sample purified using a Biotage® Isolera™ flash chromatography system equipped with a 25 g HC DUO column. The solvent system used was a ethyl acetate:acetonitrile gradient.

¹³C{1H}NMR (CD₃CN) δ152.3 151.8, 146.2, 138.6, 90.9, 55.4, 54.2, 9.4; ¹H NMR (CD₃CN) δ 7.13 (s, 1H), 4.13 (s, 3H), 3.87 (s, 3H), 2.63 (s, 3H); IR (cm⁻¹) 3228 (w), 2950 (w), 1628 (s), 1558 (m), 1496 (s), 1428 (m), 1379 (m), 1342 (m), 1326 (m), 1277 (w), 1207 (s), 1181 (m), 1071(w), 1049 (m), 947 (m), 863 (w), 764 (w), 693 (w), 632 (m), 586 (w), 524 (w), 498 (w), 432 (w); FTMS (ESI+): Expected m/z: 195.088201, Found m/z: 195.08776(Error: −2.26 ppm).

3-Isobutyl-6,8-dimethoxy-1,2,4-triazolo-[4,3-a]pyrazine (15g)

31 mg of compound (13g) was dissolved into 1 mL of acetonitrile-d3 in a borosilicate NMR tube. The solution was then irradiated for 48 hours using 254 nm UV light in a dark room. Yield determined by quantitative NMR to be 59.0%. Sample purified using a Biotage® Isolera™ flash chromatography system equipped with a 25 g HC DUO column. The solvent system used was a hexanes:ethyl acetate gradient.

¹³C{1H}NMR (CD₃CN) δ152.3, 152.0, 148.9, 138.6, 90.9, 55.4, 54.2, 32.8, 27.0, 21.6; ¹H NMR (CD₃CN) δ7.17 (s, 1H), 4.12 (s, 3H), 3.87 (s, 3H), 2.90 (d, 2H), 2.22(septet, 1H), 0.99 (d, 1H); IR (cm⁻¹): 2956(m), 2871(w), 729(w), 1678(w), 1627(m), 1554(m), 1539(m), 1492(s), 1454(m), 1428(m), 1376(m), 1322(m), 1295(m), 1275(m), 1204(s), 1181(m), 1045(s), 949(m), 862(m), 777(m), 744(m), 684(w), 633(m), 585(m), 502(w), 432(m); FTMS (ESI+): Expected m/z: 237.135151, Found m/z: 237.13480(Error: −1.48 ppm).

3-Phenyl-6,8-dimethoxy-1,2,4-triazolo-[4,3-a]pyrazine (15h)

16.5 mg of compound (13h) was dissolved into 1 mL of acetonitrile-d3 in a borosilicate NMR tube. The solution was then irradiated for 48 hours using 254 nm UV light in a dark room. Yield determined by quantitative NMR to be 61.5%. Sample purified using a Biotage® Isolera™ flash chromatography system equipped with a 25 g HC DUO column. The solvent system used was a hexanes:ethyl acetate gradient.

¹³C{1H}NMR (CD₃CN) δ153.0, 152.2, 148.6, 139.3, 130.3, 129.4, 128.0, 126.7, 91.3, 55.4, 54.5; ¹H NMR (CD₃CN) δ7.89 (d,d, 2H), 7.62 (d, 3H), 7.40 (s, 1H), 4.18 (s, 3H), 3.87 (s, 3H); IR (cm⁻¹): 2945(w), 1624(m), 1553(m), 1489(m), 1448(m), 1427(m), 1376(m), 1326(m), 1308(m), 1293(m), 1263(m), 1207(s), 1176(m), 1158(m), 1130(w), 1077(m), 1056(m), 1001(m), 947(m), 850(m), 785(s), 760(w), 722(m), 704(s), 690(m), 623(m), 612(m), 565(w), 511(m), 439(m), 423(m); FTMS (ESI+): Expected m/z: 257.103851111, Found m/z: 257.10361(Error: −0.93 ppm).

3-Cyclopropyl-6,8-dimethoxy-1,2,4-triazolo-[4,3-a]pyrazine (15i)

9 mg of compound (13i) was dissolved into 1 mL of acetonitrile-d3 in a borosilicate NMR tube. The solution was then irradiated for 48 hours using 254 nm UV light in a dark room. Yield determined by quantitative NMR to be 51.3%. Sample purified using a Biotage® Isolera™ flash chromatography system equipped with a 25 g HC DUO column. The solvent system used was a hexanes:ethyl acetate gradient.

¹³C{1H}NMR (CD₃CN) δ152.3, 151.8, 150.7, 138.7, 90.8, 55.4, 54.2, 6.0, 4.7; ¹H NMR (CD₃CN) δ7.31 (s, 1H), 4.12 (s, 3H), 3.89 (s, 3H), 1.15 (multiplet, 2H), 1.09(multiplet, 2H); IR (cm⁻¹): 2952(w), 1746(w), 1581(m), 1541(s), 1488(s), 1464(s), 1442 (m), 1424(w), 1416(m), 1396(w), 1373(s), 1348(s), 1329(s), 1283(w), 1250(m), 1239(m), 1194(s), 1176(s), 1105(s), 1064(w), 1049(w), 1017(s), 990(s), 970(m), 964(m), 920(w), 905(w), 874(m), 870(m), 777(m), 763(m), 732(s), 712(w), 700(w), 692(s), 622(w), 573(w), 561(w), 555(w), 532(w), 497(w), 471(w); FTMS (ESI+): Expected m/z: 221.103851, Found m/z: 221.10347(Error: −1.72 ppm).

3-(2,6-Difluorobenzyl)-6,8-dimethoxy-1,2,4-triazolo-[4,3-a]pyrazine (15j):

32 mg of compound (13j) was dissolved into 1 mL of acetonitrile-d3 in a borosilicate NMR tube. The solution was then irradiated for 48 hours using 254 nm UV light in a dark room. The yield determined by quantitative NMR was 42.9%. Sample purified using a Biotage® Isolera™ flash chromatography system equipped with a 25 g HC DUO column. The solvent system used was ethyl acetate:acetonitrile gradient.

¹³C{1H}NMR (CD₃CN) δ162.4(d, J=0.06), 160.4(d, J=0.07), 152.5, 151.9, 146.2, 138.9, 129.7(t, J=0.08), 111.5(d, J=0.2 ppm), 90.84, 55.3, 54.3, 18.1; ¹H NMR (CD₃CN) δ7.38 (multiplet, 1H), 7.26 (s, 1H), 7.04 (t, 2H), 4.41 (s, 2H), 4.13 (s, 3H), 3.88 (s, 3H); IR (cm⁻¹): 2999(w), 1626(m), 1564(s), 1497(s), 1439(m), 1421(m), 1375(m), 1334(m), 1313(m), 1249(m), 1206(s), 1177(m), 1073(m), 1051(m), 1031(s), 967(w), 944(s), 903(w), 881(w), 848(w), 825(w), 802(w), 750(s), 714(w), 654(m), 632(s), 506(w), 437(w); FTMS (ESI+): Expected m/z: 307.100656, Found m/z: 307.10059(Error: −0.21 ppm).

3-Bromo-8-methoxy-1,2,4-triazolo-[4,3-a]pyrazine (15b′):

13 mg of compound (13′b) was dissolved into 1 mL of acetonitrile-d3 in a borosilicate NMR tube. The solution was then irradiated for 48 hours using 254 nm UV light in a dark room. Yield determined by quantitative NMR to be 46.4%. Sample purified using a Biotage® Isolera™ flash chromatography system equipped with a 25 g HC DUO column. The solvent system used was a hexanes:ethyl acetate gradient.

¹³C{1H}NMR (CD₃CN) δ153.7, 141.5, 127.9, 122.4, 111.4, 54.3; ¹H NMR (CD₃CN) δ7.74 (d, 1H), 7.74 (d, 1H), 4.16 (s, 3H); IR (cm⁻¹): 3092 (w), 3038 (w), 2923 (w), 1731 (w), 1615 (w), 1524 (m), 1481 (m), 1455 (m), 1436 (m), 1424 (m), 1395 (m), 1374 (m), 1328 (m), 1318 (m), 1230 (s), 1181 (s), 1145 (m), 1075 (m), 1034 (m), 1014 (s), 982 (s), 957 (m), 892 (m), 794 (m), 746 (m), 727 (m), 660 (m), 629 (s), 562 (m), 516 (m); FTMS (ESI+): Expected m/z: 228.972497, Found m/z: 228.97219(Error: −1.34 ppm).

### ¹⁵N Labeled Compound (11a)

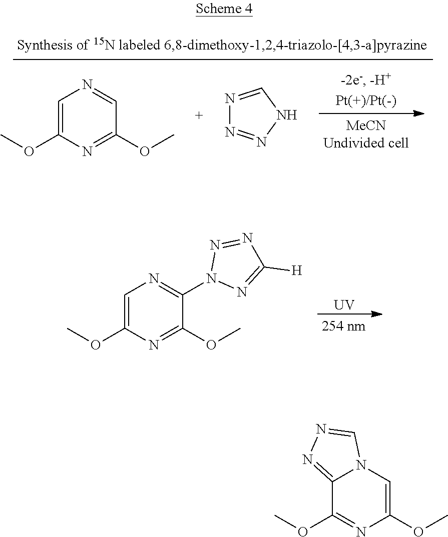

789 mg of ¹⁵N labeled sodium azide was added to a 25 mL round bottom flask with 486 mg of ¹⁵N labeled NH₄Cl and 3.5 mL of triethylorthoformate. A stir bar was then added, and the slurry was slowly stirred. 3 mL of glacial acetic acid was then added slowly in 1 mL portions. The solution was then heated to 130° C. for 24 hours. The reaction was allowed to cool to room temperature. Once cooled, 7 drops of conc. HCl was added at room temperature. The solution was then transferred dropwise to a 100 mL chilled solution of ethyl acetate. The solution was filtered to remove NaCl precipitate. The solvent was then removed by slow evaporation.

¹H NMR (CD₃CN) δ(ppm) 8.98 (tt, 1H), 8.31 (s, 2H); ¹³C{1H}NMR (CD₃CN) δ(ppm) 142.89; ¹⁵N NMR (CD₃CN) δ(ppm) 373.32(2N), δ(ppm) 278.81 (relative to NH₃).

5-nitro-[1,2,4]triazolo[4,3-a]pyrazine-6,8-diamine (27)

58 mg of compound (15a) was added slowly to 5 mL of 100% HNO₃ at 0° C. The solution immediately became a turquoise blue. The solution was allowed to stir over ice for 1 hour, followed by stirring at room temperature for 1 hour. The solution was then quenched over ice and extracted with ethyl acetate. The combined ethyl acetate fractions were dried over anhydrous MgSO₄, and the solvent was removed by rotary evaporation. 40 mL of MeOH and 80 mL of NH₄OH were then added to the flask, and the solution was heated to 100° C. for 1 hour. The solution was allowed to cool to room temperature and was then quenched over ice, which precipitated a yellow solid. This material was then collected by vacuum filtration and dried in a desiccator, which yielded 22 mg (35%) of a pure yellow solid.

¹³C{¹H}NMR (CD₃CN) δ154.0, 152.5, 140.8, 137.9, 113.9; ¹H NMR (CD₃CN) δ9.72 (s, 1H); 9.19 (s, 2H); 8.94 (s, 1H), 8.88 (a, 1H); IR (cm⁻¹): 3427(m), 3396(w), 3307(m), 3271(m), 3203(m), 3083(m), 2256(w), 1672(w), 1633(m), 1599(m), 1562(m), 1516(w), 1481(m), 1444(m), 1370(m), 1315(m), 1288(m), 1258(m), 1242(m), 1221(s), 1170(s), 1124(m), 1048(m), 1019(s), 1001(s), 985(s), 959(m), 897(m), 860(m), 823(m), 783(m), 762(m), 736(m), 718(m), 698(m), 680(m), 656(m), 595(s), 577(s), 526(s), 508(s), 482(s), 401(m); MS (ESI+): m/z: 195.9; EA: (C₇H₈N₆O₂ 195 g/mol) calcd: 50.24% N, 30.78% C, 2.58% H, found 49.51% N, 31.80% C, 2.79% H (averaged for 2 replicates); TGA (5° C. min⁻¹): 294° C. (T_{Dec, oneset}).

Electrochemical Screening Cyclic voltammetry (CV) experiments were performed using the CH Instruments 600E potentiostat. The working electrode used was a CHI 102 Pt electrode (surface area=3.14 mm²), the counter electrode was a Pt plate electrode (surface area=450 mm²), and the reference electrode was an Ag/Ag⁺ quasi-reference electrode. Before each measurement, the working electrode was polished using 1 micron Alpha Alumina powder, followed by sonication in water and acetonitrile. Following sonication, the electrodes were dried under a stream of air. The electrodes were held in a PTFE assembly to maintain electrode placement in between experiments. All measurements were performed in dry acetonitrile using 0.1 M tetraethylammonium perchlorate as the electrolyte. Samples were purged for 10 minutes with argon gas prior to the experiment. All measurements were taken under a continuous stream of argon gas to prevent oxygen intrusion. A background scan was taken of the neat solution before spiking the solution with the sample. These background scans were then subtracted from the voltammograms for the substrates of interest.

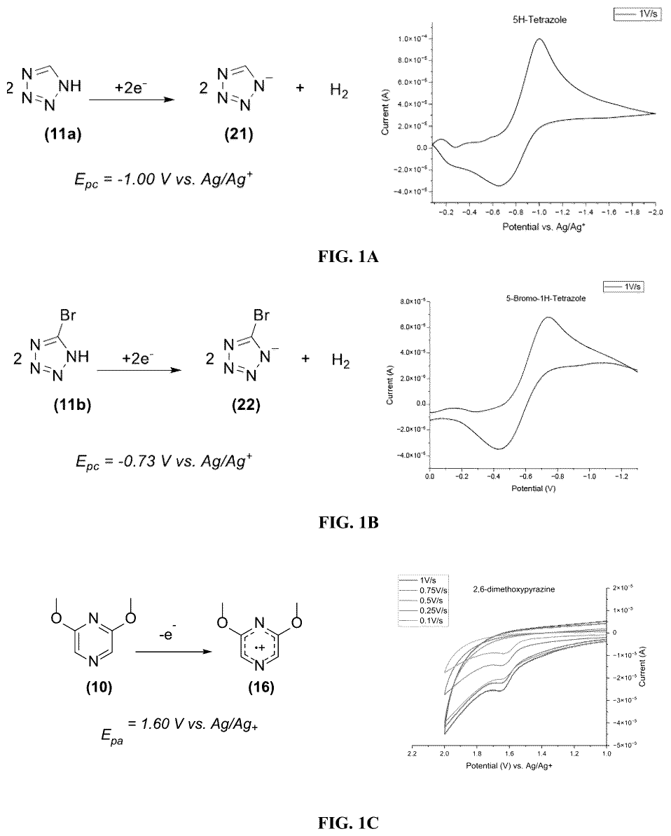

Compound (11a) is known to undergo an irreversible 2e⁻ reduction to yield a tetrazole anion (21) and hydrogen gas at platinum electrodes E_{pc}=−1.343 V vs. Fc⁺/Fc (FIG. 1a). This reduction event was observed at E_{pc}=−1.00 V vs Ag/Ag⁺ (FIG. 1a). The observed return oxidation wave corresponds to the reduction of hydrogen gas on the catalytic platinum surface. Compound (11b) showed similar redox behavior as 1H-tetrazole and, thus, undergo a similar 2e⁻ reductive pathway as a 1H-tetrazole (FIG. 1b). A peak cathodic wave was observed at −0.73 V vs. Ag/Ag⁺ which is hypothesized as the formation of a 5-bromotetrazole anion (22) (FIG. 1b). The observed return oxidation wave corresponds with the oxidation of hydrogen gas. It is hypothesized that the lowered reduction potential of compound (11b) relative to compound (11a) is due to the electron-withdrawing effects of the bromo substituent. It was found that compound (10) undergoes irreversible electrochemical oxidation at Eₚₐ=1.60 V vs. Ag/Ag⁺ (FIG. 1C, Scheme 2), which we believe corresponds to a 1e⁻ oxidation to form a radical cation (16).

Dry acetonitrile purged with argon gas was used as the solvent for all experiments. All electrolysis reactions were performed using an Ika Electrasyn 2.0 in a single cell configuration. After electrolysis, acetonitrile was removed from the reaction mixture using a rotary evaporator at 40° C. The reaction mixture was then redissolved into ethyl acetate and washed 3 times with water and once with brine. The organic layer was then dried with anhydrous magnesium sulfate, filtered, and concentrated by rotary evaporation. The dried organic layer was then purified via flash chromatography using a gradient of hexanes:ethyl acetate. Reaction yields were determined from the dried mass of products collected after purification.

The separated materials were then analyzed by LC-MS, multinuclear (¹H, ¹³C, ¹⁵N) nuclear magnetic resonance spectroscopy (NMR), infrared radiation (IR) spectroscopy, elemental analysis, differential scanning calorimetry/thermogravimetric analysis (DSC/TGA), UV Vis spectroscopy and single crystal X-ray crystallography. Gas evolution at the cathode during electrolysis indicates the likely formation of H₂ gas from the reduction of protons that are liberated from the acidic tetrazole species.

In the initial step compound (10) can be oxidized to a radical cation (16), which was prone to nucleophilic attack by tetrazole molecule. This dehydrogenative coupling resulted in an isomeric mixture of two possible radicals in which C—N bond formation can occur at either the tetrazole N1 site (17) or the tetrazole N2 site (18). Subsequent 1e⁻ oxidation of radicals (17) and (18) yielded the N1 coupled product (12a) and the N2 coupled product (13a), respectively (see Scheme 2).

The electrochemical variable screening to obtain optimal electrolysis conditions was performed using 1H-tetrazole with 2,6-dimethoxypyrazine as the model system. Results from these studies are detailed in Table 1. Reaction parameters such as species concentration, electrolyte type, anode material, cathode material, charge passed, and current were screened to determine optimal reaction conditions. The effects of current on the isolable yield were investigated. Initial electrolysis studies were performed at higher current densities (>10 mA/cm² or >25 mA/cm²), which resulted in very low product yields. Combined extracts from numerous reactions yielded a material that was identified by single crystal XRD as a dimerized species (19). The high current densities can cause an accumulation of radical species such as (18). If present in high enough concentrations, these can homolytically couple, followed by loss of the anisyl group and rearomatization, making the dimerization reaction irreversible. At lower current densities, with fewer radical species present, reduction at the cathode and loss of a proton is the more likely pathway toward rearomatization. Higher currents may also result in polymerization of the reaction mixture via this mechanism, which is supported by the observed formation of insoluble material at higher currents with a decrease in product yields. Lower currents resulted in no solid formation and an increase of the desired N2 isomer product. The effect of current on product yield is shown in Table 1, Entries 1-6. Entry 2, using a current of 7.5 mA, exhibits in the highest observed yield of the desired N2 coupled product at 16%. As a result, The current of 7.5 mA was used for the remaining electrolysis experiments.

Once determined an appropriate current density, the optimal charge passed during the electrolysis was determined (Table 1, Entries 7-11), and it was found that 2.1 F/mol of charge was ideal. At higher values of charge passed, it was observed that the product yield began to decrease with no isolable products above a charge passage of 3 F/mol. After optimizing the reaction it was observed if varied substrate concentration might impact product yields (Table 1, Entries 12-16). It was found that lowering the substrate concentrations (100 mM) resulted in lower yields of both coupled isomers. This may be due to a decreased probability for coupling to occur between electrochemically generated reactants. Next, the parameter investigated was how changing the solution pH may impact product yields (Table 1, Entries 17-18). Almost identical yields for both coupled isomers with and without the addition of collidine were observed. Next, the contribution of electrolytes to the reaction yield was tested (Table 1, Entries 19-22). Upon the addition of LiClO4 to a solution of 2,6-dimethoxybenzene and 1H-tetrazole, a white precipitate crashed out of the solution. Electrolysis of this slurry results in no yield of the desired product. It was hypothesized that a double displacement reaction occurs between lithium perchlorate and 1H-tetrazole to yield a perchlorate anion, a hydronium cation, and lithium tetrazolate. Tetraethylammonium tetrafluoroborate (Table 1, Entry 20) was found to be a suitable electrolyte with marginally lower yields than tetraethylammonium perchlorate. The use of iodine-mediated electrolysis (Table 1, Entry 21) was unsuccessful, with no detectable formation of the N2 couple. The most successful electrolyte, and the most successful overall reaction conditions involved the use of tetraethylammonium tetrazolate. This electrolyte was prepared by combining a stoichiometric amount of 5H-tetrazole with tetraethylammonium hydroxide, followed by crystallization. For the subsequent scope experiments, tetraethylammonium perchlorate was used as the electrolyte to aid in controlling anhydrous conditions due to the deliquescence of tetraethylammonium tetrazolate salts. The effect of electrode material on the electrolysis reaction was tested. Use of graphite rather than platinum for the cathode material resulted in a significant decrease in product formation (Table 1, Entry 23). Varying the cathode materials to stainless steel (SS) or nickel also resulted in a significant decrease in product yield (Table 1, Entries 24-25).

Once the suitable electrolysis reaction conditions were determined, they were then applied towards the coupling of (10) and (11b) (Table 2) and applied to various tetrazole species (11a-q) (Scheme 5). Initial experiments yielded very low product formation (Table 2, Entry 1). Doubling the current of the reaction to 15 mA significantly improved the formation of the desired product, with a close to 50% yield of the N2 couple 15b (Table 2, Entry 3).

5-bromotetrazole (11b) was used for future coupling reactions due to its ease of synthesis that avoids highly dangerous hydrazoic acid, its more sterically hindered and electronically deactivated N1 site, and its potential for further downstream functionalization afforded by halogen substitution reactions (New Journal of Chemistry, 2022, 46, 21085-21091). Additionally, 5-bromoterazole proved advantageous for the detection of coupled materials via LC-MS analysis due to the characteristic M/Z signature resulting from the isotopic nature of bromine. Lastly, 5-bromotetrazole was found to couple with heterocycles that 5H-tetrazole failed to show coupling (e.g., 2-methoxypyrazine and 2-chloro-6-methoxy pyrazine). The electrochemical coupling of (10) with (11a-q) generally resulted in low isolated yields of the desired N2 coupled products (13a-q) under standard reaction conditions. Initial experiments to test the scope of methodology were performed using halogen-bearing bromo tetrazole (11b). Under standard conditions, this electrolysis reaction results in a roughly 1:1 mol ratio (13.1% yield 12b and 12.7% yield 13b). The lower yield of N2 couple (13b) (12.7%) relative to (13a) (16.1%) under identical reaction conditions supports the hypothesis for the hydrogen evolution reaction (HER) reaction occurring at the cathode. This conclusion was supported by cyclic voltammetry studies of (11a) and (11b) (FIG. 1A and FIG. 1B). Compound (11a) is known to undergo an irreversible 2e-reduction to yield a tetrazole anion (21) and hydrogen gas at platinum electrodes Epc=−1.343 V vs. Fc+/Fc. This reduction event was observed at Epc=−1.00 V vs Ag/Ag+(FIG. 1A). The observed return oxidation wave corresponds to the reduction of hydrogen gas on the catalytic platinum surface. A peak cathodic wave of (11b) was observed at −0.73 V vs. Ag/Ag+ which is hypothesized as the formation of a 5-bromotetrazole anion (22) (FIG. 1B). The observed return oxidation wave corresponds with the oxidation of hydrogen gas. If the cathodic half-reaction was tetrazolate formation, then it would be expected that (11b) would show an improved reaction efficiency when compared to (11a) due to its more positive reduction potential. Surprisingly, it was found that a higher current of 15 mA significantly and selectively improved the formation of the desired isomer (13b) with a slight improvement to the formation of the undesired N1 isomer (38.8% yield 13b, 17.7% yield 12b) (Table 2, Entry 2). This implies that electrolysis conditions are highly dependent on the electronic nature of the tetrazole species in use. The use of tetraethylammonium bromotetrazolate as the electrolyte was found to improve reaction efficiency even further with an isolated yield of 49.5% (13b) and 24.3% (12b).

5-Azidotetrazole (11c) was selected to demonstrate potential energetic scope. It is important to emphasize the potential hazards of working with (11c) as the free acid is a highly sensitive and high-performing energetic material (Klapötke, J Am Chem Soc 2009, 131, 1122-11340). Due to the hazards involved, compound (11c) was immediately used for electrolysis following its synthesis without further sample manipulation or purification. This was performed by combining 1 molar equivalent of 2,6-dimethoxypyrazine and anhydrous acetonitrile with the freshly prepared compound (11c) after it has been dried and weighed in a plastic apparatus. This made it nearly impossible to perform these reactions at a 2 mmol scale due to the variability in yield of compound (11c). As a result of performing these electrolysis reactions on crude compound (11c) it was discovered that the standard literature procedure for the synthesis of compound (11c) results in a significant amount of 5-methoxytetrazole (11d) formation as a byproduct of the reaction (Klapötke, J Am Chem Soc 2009, 131, 1122-11340). This was determined to be the result of use of methanol in the literature procedure. Removal of methanol from the 5-azidotetrazole synthesis resulted in the selective formation of 5-azidotetrazole. The electrolysis of compound (11c) with compound (10) under standard reaction conditions resulted in the formation of compound (12c) and compound (13c) at 18.6% and 27.2%, respectively. Additionally, it was found that compound (12n) was also produced during this electrolysis reaction in a 3% yield. It was believed that the amine formed due to the cathodic electrolysis of the azide functional group on compound (12c) in the single-compartment configuration. A single crystal of this material was obtained from the slow evaporation of solvent following column purification. This was a potentially advantageous means to acquire compound (12n) and compound (13n) due to the poor solubility of 5-aminotetrazole that greatly hinders coupling with the compound (10). Compound (13n) was not isolated or detected from the electrolysis reaction of compound (11c) with compound (10).

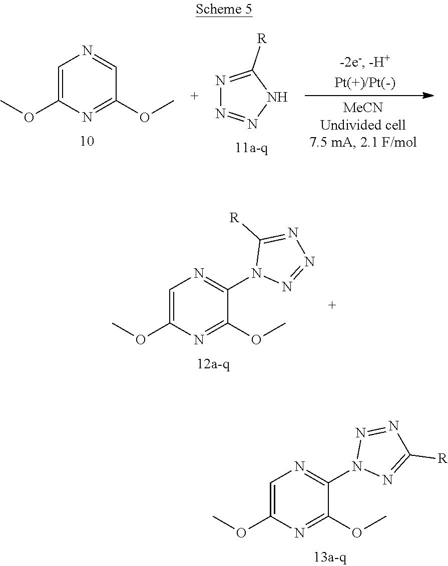

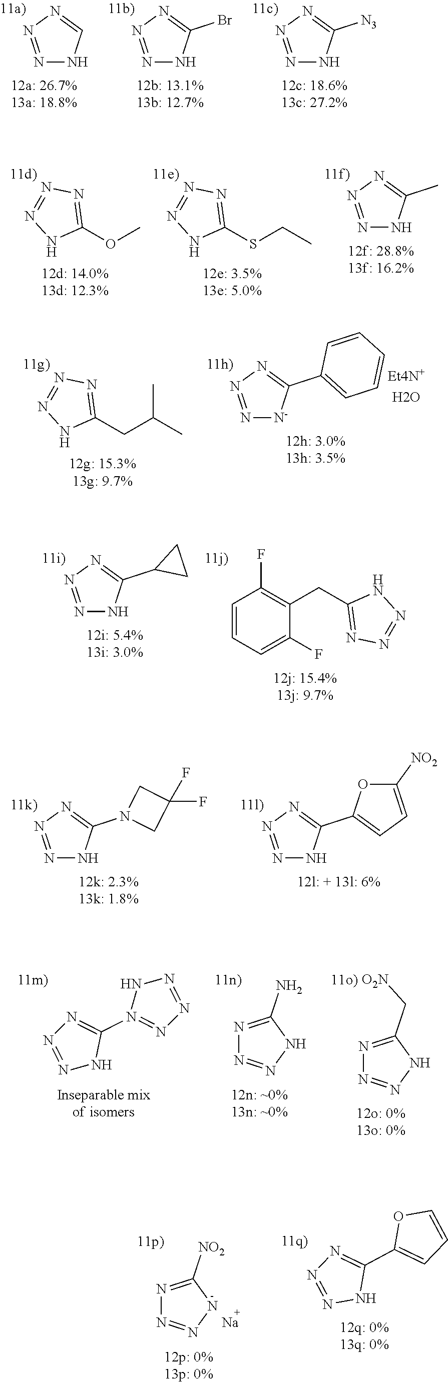

The attempts were made to selectively form compound (11d) for use in coupling reactions. Cyanogen bromide was allowed to stir in a solution of water and methanol for 24-48 hours prior to addition of sodium azide in an attempt to force a reaction between methanol and cyanogen bromide. These prolonged experiments still produced significant quantities of azidotetraozle (˜80%). Addition of base, such as triethylamine or sodium carbonate, to neutralize HBr formed from methanol addition to cyanogen bromide resulted in no yield of either compound (11c) or compound (11d). It was found that compound (11d) can not produced selectively. The coupling reaction of compound (11d) and compound (10) was performed with the presence of compound (11c). Due to the presence of compound (11c) in the reaction mixture, the sample was used as is without further purification prior to electrolysis to minimize safety hazards from the handling of compound (11c). Quantitative NMR analysis of the reaction mixture indicated a mass percentage of 21.2% compound (11d) (0.43 mmol) and 78.4% (1.41 mmol) compound (11c) within a 200 mg sample. This reaction mixture was combined with 2 mmol of 2,6-dimethoxypyrazine, and electrolysis was performed under standard conditions. This electrolysis reaction resulted in the formation of compounds (12c), (13c), (12d), and (13d), at 7%, 3.2%, 14.0%, and 12.3%, respectively, relative to mol of tetrazole starting material. From these results, it may be observed that the presence of compound (11d) at low concentrations suppresses the coupling of compound (11c) with compound (10). The effect of other non-bulky electron donor substituents at the tetrazole C5 position on electrochemical coupling was studied. The electrolysis of 5-ethyltetrazole (11e) with compound (10) resulted in the formation of compound (12e) and compound (13e) at 3.5% and 5.0%, respectively. Purification of the crude reaction mixture following electrolysis reveals that approximately 65% of the compound (11e) was unreacted. Trace amounts of compound (10) were detected that indicated consumption via unwanted side reactions. 5-methyl tetrazole (11f) was coupled with compound (10) to form compound (12f) and compound (13f) at 28.8% and 16.2% respectively. LC-MS analysis of highly polar phases from this coupling reaction reveals likely di-addition of compound (11e) to compound (10). This implies that the mono-coupled products are prone to electrochemical oxidation and addition of a second tetrazole species. The compound (11g) was found to couple with compound (10) to form compound (12g) and compound (13g) at 15.3% and 9.7%, respectively. Additionally, LC-MS results indicate the presence of di-coupling of compound (11g) to compound (10). The coupling of compound (11h) with compound (10) resulted in a nearly 1:1 mol ratio of compound (12h) and compound (13h) with a 3.0% yield and 3.5% yield, respectively. It is important to note that no reaction was observed when using the free base form of the compound (11h) due to poor solubility in anhydrous acetonitrile. The tetraethylammonium salt of compound (11h) was prepared by combining compound (11h) free acid with 1.0 eq. of tetraethylammonium hydroxide. Subsequent drying of this material in a P₂O₅ desiccator yields 5-phenyltetrazolate tetraethylammonium monohydrate salt. The low reaction efficiency is possibly attributed to the presence of water of hydration on 5-phentyltetrazolate salt.

5-Cyclopropyl tetrazole (11i) was found to couple with compound (10) to form compound (12i) and compound (13i) at a 5.4% yield and a 3.0% yield, respectively. 5-difluorobenzyltetrazole (11j) was found to couple with compound (10) to form compound (12j) and compound (13j) at a 15.4% yield a 9.7% yield respectively. 5-difluoroazetidine-tetrazole (11k) was found to couple with compound (10) to form compound (12k) and compound (13k) at a 2.3% yield and a 1.8% yield, respectively. These 3-ring substituted tetrazole molecules exhibit preferential addition to the more sterically hindered N1 site, which indicates coupling is dominated by the electronic effects of the substituent on the tetrazole species. The low yields from using compound (11i) and compound (11k) are likely due to the decomposition of the ring-strained cyclopropyl and difluoroazetidine. No starting materials were recovered following the electrolysis reactions involving these cyclic substituted tetrazole species, which further supports this hypothesis. 5-(2-nitrofuran)-tetrazole (11l) was found to successfully couple to 2,6-dimethoxypyrazine; however, the isolated material was found to be an inseparable mixture of both the N1 and N2 isomer at an overall yield of 6%. Successful coupling of species 5,5′-bistetrazole (11m) and 5-aminotetrazole (11n) was observed via electrospray ionization (ESI) High Resolution Mass Spectrometry (Hi-Res MS). However, both tetrazoles had individual difficulties with isolation. Due to the multiple bonding sites present on the compound (11m), an inseparable mixture of isomers was collected following electrolysis, which precluded further workup and yield determination. Compound (11q) is poorly soluble in acetonitrile as both the free acid and tetraethylammonium salt.

### Thermal Analysis

The desired nitrilimine intermediate may also be obtained by thermally heating compounds (13a) and (13b). The enthalpy of formation of this nitrilimine intermediate can be determined experimentally by measurement of the endothermic transition via DSC/TGA. Compound (13a) has an endothermic onset at 67° C. with an enthalpy of 115 J/g. Compound (13b) has an endothermic onset at 81° C. with a lower enthalpy of 71 J/g. This observed a decrease in enthalpy of formation due to bromine stabilization of the nitrilimine. Nitrilimines exist in 6 possible structures depending on the substituents: propargylic, allenic, carbenic, diradical, 1,3-dipolar, and reverse 1,3-dipolar. The substitution of the C terminus of the nitrilimine with a heteroatom is known to increase the contribution of the carbenic resonance structure. This additional carbenic character can lower the energy barrier for nitrilimine formation, hence the lower observed enthalpy of (13b). The onset of decomposition was determined from these studies from the onset of the first exothermic event. Compounds (13a) and (13b) begin to decompose at 139° C. and 179° C., respectively. Compounds (15a) and (15b) lack any endothermic transition states and decompose at 155° C., and 147° C., respectively.

1,5-disubstituted tetrazoles, such as compounds (12a) and (12b), have endothermic transition states that correspond to a nitrene formation. Compound (12a) has an endothermic onset at 101° C. with an enthalpy of 137 J/g. Compound (12b) has an endothermic onset at 65° C. with an enthalpy of 75 J/g. This observed decrease in enthalpy may be attributed to the electronic stabilization of the nitrene due to the adjacent bromine atom. Compounds (12a) and (12b) begin to decompose at 150° C., and 149° C., respectively.

Introducing substituents that are capable of inter and intramolecular hydrogen bonding, such as amine and nitro substituents, increases decomposition temperature. High decomposition temperature is often necessary for the practical handling of energetic materials.

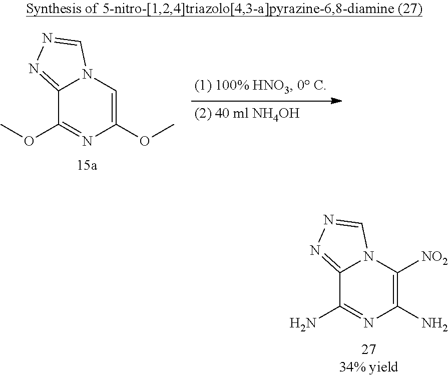

Nitro group was introduced by nitrating compound (15a) in white fuming nitric acid followed by substitution of the methoxy by amino groups by reflux in MeOH:NH₃(aq)(28%) (1:2) for 1 hour (Scheme 4). After reflux, the solution was allowed to cool to room temperature and then quenched over ice, which precipitated a yellow powder that was identified as 5-nitro-[1,2,4]triazolo[4,3-a]pyrazine-6,8-diamine (27). The structure and purity of compound (27) were determined by multinuclear NMR, LC-MS, and elemental analysis. The crystalline density and heats of formation of 20 were determined using the Gaussian09 program package. The geometry of 20 was initially optimized using the spin-restricted B3LYP density functional theory with the 6-31G** basis set. The presence of energetic minima was verified by the absence of negative vibrational frequencies. Theoretical densities were calculated using the methods of Byrd and Rice. The energy of formation was obtained using the electronic energies from a calculation at the B3LYP/6-311++G(2df, 2p) level of theory and electrostatic parameters on the 0.001 electron/bohr3 isosurface. The energetic performance of compound (27) was then calculated from these theoretical heats of formation and crystalline density using the software package EXPLO5®. The predicted energetic performance of compound (27) is quite low, with a detonation velocity of 7222 m/s and a detonation pressure of 18.2 GPa. However, the thermal decomposition temperature appears to be promising. The onset of decomposition was determined to be 294° C. by TGA (FIG. 2).

TABLE 3
Energetic performance of compound (27)

| | |
|---|---|
| ρ_{calc} ^{a} (g cm⁻³) | 1.752 |
| H_{f}^{b} (kcal/mol) | 46.444 |
| −Δ_{Ex}U^{c} (kJkg⁻¹) | −2896.84 |
| T_{Det}^{d} (K) | 294 |
| P_{CJ}^{e}/GPa | 18.5 |
| V_{Det}.^{f}/m s⁻¹ | 7222 |
| Vₒ^{g}/L kg⁻¹ | 712.5 |

### Spectroscopy Analysis

A weak IR absorption band corresponding to a methoxy group was observed for all compounds at wavelengths 2860-2800 cm⁻¹. The pyrazine ring undergoes average C—C bond vibration at 1322 cm⁻¹ and average C—N bond vibration at 1221 cm⁻¹. An absorption band associated with tetrazole compounds is seen at −1240 cm⁻¹. ¹⁵N labeled 1H-tetrazole was prepared from ¹⁵N labeled ammonium chloride and ¹⁵N labeled sodium azide to aid in the confirmation of the proposed mechanism. This ¹⁵N labeled 11a was then used to synthesize ¹⁵N labeled compounds (13a) and (15a) (Scheme 4). The ¹⁵N NMR spectra of ¹⁵N labeled compound (11a) measured in CD₃CN-d3 show 2 signals (N(2), δ=−7.2; N(2), δ=−101.7) that correspond to the four nitrogen atoms of the tetrazole ring. After electrolysis, the vertical symmetry of the tetrazole molecule was lost due to covalent bonding to the pyrazine backbone, as a result, the ¹⁵N spectra of compound (13a) showed four distinct peaks that correspond to the four nitrogen atoms of the tetrazole ring (1, 8=2.9; 1, 8=−45.3; 1, 8=−72.6; 1, δ=−97.6). Photolysis of compound (13a) resulted in the loss of N₂ and N₄ from the tetrazole ring as nitrogen gas. The ¹⁵N spectra of compound (15a) showed two distinct signals corresponding to the two nitrogen atoms on the triazolo ring (δ=−46.2; 1, δ=−71.4).

### UV Cyclization

2,5-disubstituted tetrazole compounds such as (13a) and (13b) were dissolved in CH₃CN and irradiated using a handheld 254/365 nm UVP UVGL-55 lamp in quartz test tubes. Thin layer chromatography (TLC) UV lamp was used as a UV source, which was placed into a steel container with reflective sides and covered with a lid during operation, ensuring direct exposure of the compound solution to UV light. UV irradiation of the solution was then performed for 48 hours using 254 nm UV light. The extent of the reaction was monitored by ¹H NMR.

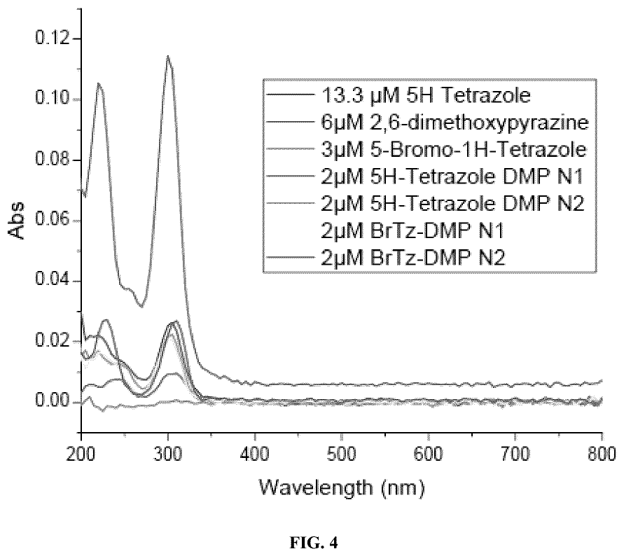

It was found that both 254 nm and 365 nm are effective wavelengths for nitrilimine formation. After 24 hours of irradiation, nearly all the original compound (13b) was consumed using either 254 nm or 365 nm UV light. It was observed that excessive irradiation results in the decomposition of compound (15b), with no detectable peak after four days using 254 nm UV light (FIG. 4) and only 2 days using 365 nm UV light. For this reason, the extent of the reaction was limited to 48 hours using 254 nm UV light. Compounds (13b), (13e), (13g), and (13′b) were found to undergo complete conversion to the desired 3-substituted 1,2,4-triazolo-[4,3-a]pyrazine after 48 hours.

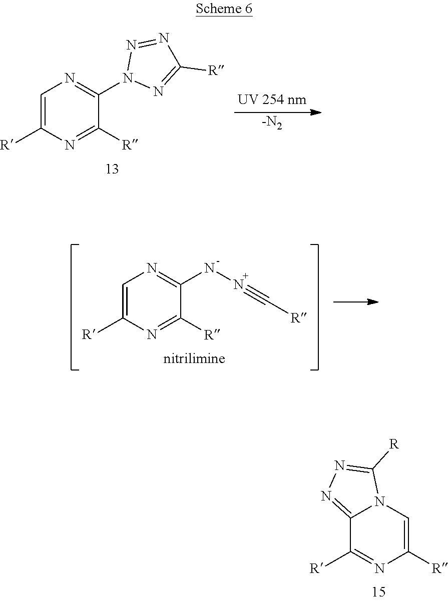

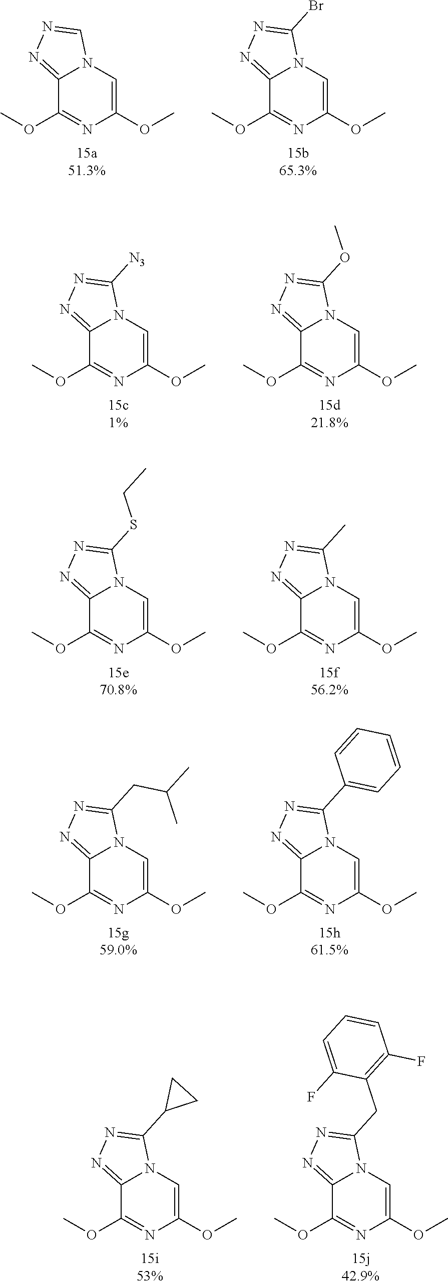

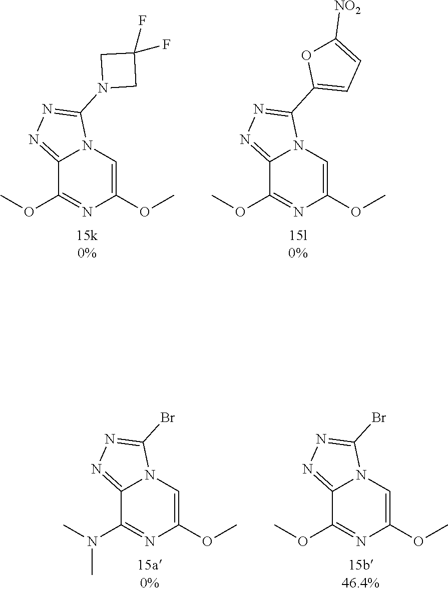

The UV-VIS absorption studies revealed that the coupled compounds (13a) and (13b) absorption spectra closely resemble compound (10) but are slightly redshifted (FIG. 4). Compound (13a) also displays an additional absorption band at 250 nm. It is known that pyrazine undergoes UV photoisomerization to pyridazine and bond cleavage to release HCN. However, the shorter 254 nm UV light decreases the absorption of UV light by the pyrazine ring and decreases the rate of decomposition/photoisomerization. The reaction yields were determined from quantitative NMR analysis using 1,3,5-trimethoxybenzene as an internal standard following 48 hours of UV irradiation. Results indicated compound (13a) is converted to compound (15a) in a 51.3% yield (Scheme 6), whereas compound (13b) is converted to compound (15b) in a 65.3% yield (Scheme 6).

Due to the nature of the highly reactive nitrilimine intermediate formed under UV light, certain substituents present on either the tetrazole or pyrazine ring either prevented cyclization from occurring or significantly decreased yields relative to other materials. Amine-bearing 2,5-disubstituted tetrazole, such as compounds (13k) and (13′a) decomposed completely under UV exposure, suggesting that the amine interacts preferentially with the highly reactive nitrilimine. Low yields were also observed for the UV exposure of compounds (13c) and (13i). This is likely due to unwanted interactions of azido group and the ring-strained cyclopropyl group with the UV-generated nitrilimine. Other 2,5-disubstituted tetrazole species, such as compound (13f), were found to undergo incomplete conversion with large amounts of starting material present within the reaction mixture. The unseparated mixture of compounds (12l) and (13l) was found to be completely unaffected by UV exposure.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid the reading of the document and is not to be interpreted as limiting. Further, information that is relevant to a section heading may occur within or outside of that particular section. The terms “including” and “having” are defined as comprising (i.e., open language).

Those skilled in the art will recognize that numerous modifications can be made to the specific implementations described above. The implementations should not be limited to the particular limitations described. Other implementations may be possible. While the inventions have been illustrated and described in detail in the drawings and foregoing description, the same is to be considered illustrative and not restrictive in character, it being understood that only certain embodiments have been shown and described and that all changes and modifications that come within the spirit of the invention are desired to be protected.

We claim:

1. A method for preparing a 1,2,4-triazolo-[4,3-a]pyrazine of formula (IV): 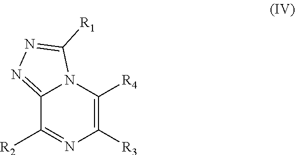
wherein,
each R₁, R₂, R₃, and R₄ is independently hydrogen, halogen, nitrile, azide, nitro, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X is O or S;
each R₅ and R₆ is independently hydrogen or a substituted or an unsubstituted group selected from alkyl, trifluoroalkyl, cycloalkyl, hydroxyl, alkoxy, thiol, thioalkyl, amine, aminoalkyl, carboxy, aminocarbonyl, N-alkyl aminocarbonyl, N,N-dialkyl aminocarbonyl, heteroalkyl, heterocycloalkyl, aryl, heteroaryl, heteroarylalkyl, heteroalkylaryl, arylalkenyl, and arylalkyl;
which method comprises:
(i) mixing a compound of formula (I): 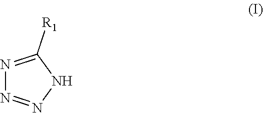
wherein,
R₁ is independently hydrogen, halogen, nitrile, azide, nitro, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X is O or S;
each R₅ and R₆ is independently hydrogen or a substituted or an unsubstituted group selected from alkyl, trifluoroalkyl, cycloalkyl, hydroxyl, alkoxy, thiol, thioalkyl, amine, aminoalkyl, carboxy, aminocarbonyl, N-alkyl aminocarbonyl, N,N-dialkyl aminocarbonyl, heteroalkyl, heterocycloalkyl, aryl, heteroaryl, heteroarylalkyl, heteroalkylaryl, arylalkenyl, and arylalkyl;
with a compound of formula (II): 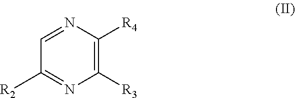
wherein,
each R₂, R₃, and R₄ is independently hydrogen, halogen, nitrile, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, aryl, heteroaryl, heteroarylalkyl, heteroalkylaryl, arylalkenyl, and arylalkyl, wherein X, R₅, and R₆ are as defined above;
in the presence of an organic solvent and an electrolyte to obtain a reaction mixture;
(ii) subjecting the reaction mixture of step (i) to an electrolysis to obtain a compound of formula (III): 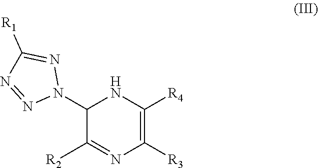
wherein,
each R₁, R₂, R₃, and R₄ is independently hydrogen, halogen, nitrile, azide, nitro, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, heterocyclyl, aryl, heteroaryl, heteroarylalkyl, heteroalkylaryl, arylalkenyl, and arylalkyl, wherein X, R₅, and R₆ are as defined above;
and
(iii) subjecting the compound of formula (III) to a photochemical reaction using an ultraviolet (UV) light,
whereupon 1,2,4-triazolo-[4,3-a]pyrazine of formula (IV) is obtained.

2. The method of claim 1, wherein R₁ is hydrogen or halogen.

3. The method of claim 1, wherein R₁ is hydrogen, halogen, azide (N₃), nitro or a substituted or an unsubstituted group selected from alkyl, cycloalkyl, OR₅, SR₅, NR₅R₆, aryl, and heterocyclyl, wherein each R₅ and R₆ is independently selected from hydrogen, alkyl, trifluoroalkyl, cycloalkyl, aryl, heteroaryl, heterocyclyl, heteroarylalkyl, heteroalkylaryl, arylalkenyl, and arylalkyl.

4. The method of claim 3, wherein R₁ is selected from hydrogen, halogen, N₃, OMe, S-Et, methyl, isovaleryl, cyclopropyl, NO₂, CH₂—NO₂, NH₂, Phenyl, tetrazole, furan, 2-nitro furan, benzyl, difluoro benzyl and difluoroazetidine.

5. The method of claim 1, wherein R₄ is hydrogen.

6. The method of claim 1, wherein the compound of formula (II) is a compound of formula (IIA): 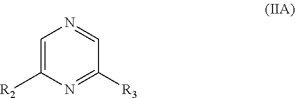
wherein,
each R₂ and R₃ is independently hydrogen, halogen, nitrile, or a substituted or an unsubstituted group selected from alkyl, trifluoroalkyl, cycloalkyl, X—R₅, NR₅R₆, COOR₅, COR₅, COONR₅R₆, SO₂R₅, heteroalkyl, heterocycloalkyl, aryl, heteroaryl, heteroarylalkyl, heteroalkylaryl, arylalkenyl, and arylalkyl, wherein X is O or S;
each R₅ and R₆ is independently hydrogen or a substituted or an unsubstituted group selected from alkyl, trifluoroalkyl, cycloalkyl, hydroxyl, alkoxy, thiol, thioalkyl, amine, aminoalkyl, carboxy, aminocarbonyl, N-alkyl aminocarbonyl, N,N-dialkyl aminocarbonyl, heteroalkyl, heterocycloalkyl, aryl, heteroaryl, heteroarylalkyl, heteroalkylaryl, arylalkenyl, and arylalkyl.

7. The method of claim 6, wherein each R₂ and R₃ is independently hydrogen, halogen or a substituted or an unsubstituted group selected from alkyl and OR₅, wherein R₅ is alkyl, trifluoroalkyl, cycloalkyl, aryl, heteroaryl, heterocyclyl, heteroarylalkyl, heteroalkylaryl, arylalkenyl, or arylalkyl.

8. The method of claim 1, wherein the organic solvent is a polar aprotic organic solvent.

9. The method of claim 1, wherein the electrolyte is selected from tetraalkylammonium salts, trialkyl ammonium salts, tetraalkylammonium tetrazolate, organic or inorganic perchlorates, organic or inorganic tetrafluoroborates, organic or inorganic nitrates, and organic or inorganic hexafluorophosphates.

10. The method of claim 9, wherein the electrolyte is selected from tetraethylammonium tetrafluoroborate, sodium tetrafluoroborate, sodium perchlorate, tetraethylammonium perchlorate, tetrapropylammonium perchlorate, tetraethylammonium tetrazolate, tetraethylammonium bromo tetrazolate, tetrabutylammonium hexafluorophosphate and lithium nitrate.

11. The method of claim 1, wherein the photochemical reaction can be carried out using an organic solvent.

12. The method of claim 11, wherein the organic solvent is a polar aprotic organic solvent.

13. The method of claim 1, wherein the photochemical reaction is carried out using a UV light having a wavelength of about 155 nm to about 465 nm.

14. The method of claim 13, wherein the photochemical reaction is carried out using a UV light having a wavelength of about 250 nm to about 370 nm.

15. A method for synthesizing an energetic material 5-nitro-[1,2,4]triazolo[4,3-a]pyrazine-6,8-diamine of formula (27), which method comprises: 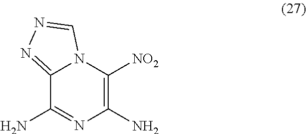 [SMILES: Nc1nc(N)c2nncn2c1[N+](=O)[O-]]
(i) preparing 6,8-dimethoxy-1,2,4-triazolo-[4,3-a]pyrazine a compound of formula (15a), wherein a compound of formula (15a) is prepared according to the method of claim 1; and 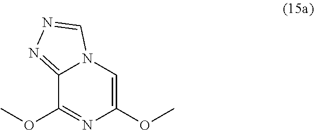
(ii) introducing a nitro and amino group to the compound of formula (15a), whereupon the energetic material 5-nitro-[1,2,4]triazolo[4,3-a]pyrazine-6,8-diamine of formula (27) is synthesized.
